SEQUENCE LISTING (52 sequences)

SEQ ID NO: 1 - DNA, 1353 bp, Homo sapiens
caggctattccggacgccagccctggaagctgagcctgacccagcaggtcccagcagcctggcagcccggccagcatgcagcagcagcctctgcccgggcctggcgcccccacaactgagccaaccaagcctccctacagctacatcgcccttattgctatggccatccagagctcaccggggcagcgggccaccctcagtggcatctaccgctacatcatgggccgattcgccttctaccgccacaaccggcccggctggcagaacagcatccgccacaacctgtcactcaacgagtgctttgtcaaggtgccccgcgatgaccgcaagccaggcaagggcagctactggacgctggaccctgactgccacgacatgtttgagcacggcagcttcctacgccgccgccgccgcttcacccggcagacaggtgctgagggcacccggggccccgccaaggcacgccgtggacccctcagggcgaccagccaggacccaggagtccccaacgccacgaccggcaggcagtgctcattcccaccagagctgccagatcccaagggcctaagctttgggggtctggtgggggccatgccagccagtatgtgcccagcaaccactgatggcaggcctcggccacccatggagcccaaagagatttccacgcccaagcctgcatgcccaggggagctccccgtggccacctcatcttcctcatgcccagcgtttggctttcctgccggcttctcagaggctgagagttttaataaggcccctacgcccgtcttgtccccggaatcaggcatcgggagcagctaccagtgtcggctgcaggcactgaatttttgcatgggggctgacccaggccttgagcacctcttggcctcagcagccccctcccctgcaccacccacccctccaggctcactccgggccccactgcccctgccaactgaccacaaggaaccctgggttgcaggtggcttccctgtccagggaggctccggctacccattggggctgaccccctgcctataccggacgccaggaatgttcttctttgagtaaaggcagcctcacctcgggcagtccctgcaggtccctcaccctccgggctgagcctggctctaggacctgaagaactctcgaaggacccagccctggtgagccaaggacaaccacacagaaagccaggattgaagcggtgctcagccaggcccctggggcctccggacaaacttgggggtgaggggaagcagggcctcttgggatttactctgtggctctcagggccaataaagccatgtgatgatgagggaaaaaaaaaaaaaaaaaaaaaaaaaaaaaaaaaaa

SEQ ID NO: 2 - protein, 330 aa, Homo sapiens
MQQQPLPGPGAPTTEPTKPPYSYIALIAMAIQSSPGQRATLSGIYRYIMGRFAFYRHNRPGWQNSIRHNLSLNECFVKVPRDDRKPGKGSYWTLDPDCHDMFEHGSFLRRRRRFTRQTGAEGTRGPAKARRGPLRATSQDPGVPNATTGRQCSFPPELPDPKGLSFGGLVGAMPASMCPATTDGRPRPPMEPKEISTPKPACPGELPVATSSSSCPAFGFPAGFSEAESFNKAPTPVLSPESGIGSSYQCRLQALNFCMGADPGLEHLLASAAPSPAPPTPPGSLRAPLPLPTDHKEPWVAGGFPVQGGSGYPLGLTPCLYRTPGMFFFE

SEQ ID NO: 3 - DNA, 1311 bp, Mus musculus
aacagcctgggcagcctgtgcagatcgccagctctgaataccgtgcctggctttgcggtgtcagcaacacagccagcatgcagaagcaaccatcaccagaatccttggctccttctgcagagcccaccaagcccccttacagctacatagccctgattgccatggctatccagagttcaccgggtcagcgggccaccctcagtggcatctaccgctacatcatgggccgctttgccttttaccggcacaaccggccgggctggcaaaacagcatccgccataacctgtctctcaacgagtgtttcgtcaaggtgcctcgtgatgaccgcaagccaggcaagggcagctactggaccctggatccggactgccacgacatgttccagcatggcagctttctccgtcgccgcagacgcttcaccaaacgcacaggtgcgcagggcaccaagggccctgtcaagatagaccacagaccccacagagccaccagcccggacccaggagcccccaagaccacaactggcagactgtgcccattcccacaagaggtgccaaaccccaagggcttaagctttgagggtctgatggggtctttgccagccaacatgagctcaacaaccagtgacgtcaggccccaactgcccactggacccaaagagatgtgctcggcaaagtctggaggcccaagggagctctctgaagctacctcgccctcaccgtgcccagcattcggcttttcatctgccttctctgacgctgagagtctcggcaaggcccccacacccggtgtggctcctgagagcgttgggagcagctaccagtgccggatgcagacactgaatttctgtatggggactgacccaggccttgaacacctcctggtctcttcagtccccacccctggatcctcgacaccttcagcctctcaccgggccccactgcccctgccggctgactccaaagaaccctgggtggctgggagcttccctgtccagggaggttctggctatccactgggattacccccctgtctttaccggactccgggaatgttcttcttcgagtgattggctgcagcggcccctctgtagggctcctgtcaactgttctctggagactcagactggctatatgacctagggcactctgcaagaacccagccctggcaggccaaggactgtgaggatcagaaggcagaagtgagcagtcaggcaggtcccttgggggcctcctgacaaacttgggatgtggggaaatggaacccttaagcttttctctgtggttctcagggctaataaagccggtgtggtg

SEQ ID NO: 4 - protein, 329 aa, Mus musculus
MQKQPSPESLAPSAEPTKPPYSYIALIAMAIQSSPGQRATLSGIYRYIMGRFAFYRHNRPGWQNSIRHNLSLNECFVKVPRDDRKPGKGSYWTLDPDCHDMFQHGSFLRRRRRFTKRTGAQGTKGPVKIDHRPHRATSPDPGAPKTTTGRLCPFPQEVPNPKGLSFEGLMGSLPANMSSTTSDVRPQLPTGPKEMCSAKSGGPRELSEATSPSPCPAFGFSSAFSDAESLGKAPTPGVAPESVGSSYQCRMQTLNFCMGTDPGLEHLLVSSVPTPGSSTPSASHRAPLPLPADSKEPWVAGSFPVQGGSGYPLGLPPCLYRTPGMFFFE

SEQ ID NO: 5 - DNA, 1316 bp, Mucaca mulata
caggctgttccggacgtgagccctggacgccgagcctgacccagcaggtcctggcagcccggccagcccggccagcatgcagcagcagcctctgcccgggcccggggcccctgcagctgagccaaccaagcctccctacagctacatcgcccttattgccatggccatccagagctcgccggggcagcgggccacgctcagtggcatctaccgctacatcatgggccgcttcgccttctaccgccacaaccggcccggctggcagaacagcatccgccacaacctgtcactcaacgagtgctttgtcaaggtgccccgcgatgaccgcaagccaggcaagggcagctactggacgctggaccctgactgccacgacatgtttgagcacggcagcttcctacgccgccgccgccgcttcacccggcggacaggtgctgagggcaccaggggcccggccaaggcacgccgtggacccctcagggcaaccagccaggacccaggagtcctcgacaccacgaccagcaggcagtgctcgttcccaccagagctgccagaccccaagggcctaagctttgggggtctggtgggggccatgccagccagtatgtgcccagcaaccactgatggcaggcctcggccacccacggagcccaaagagatttccacgcccaagcctgcatgcctgggggagctccccgtggccacctcatcttcctcatgcccagtgtttggctttcctgccggcttctcggaggctgggggttttaataaggcccctacacccgtctcgtccccggaagcaagcatcgggagcagctaccagtgtcggctgcaggcactgaatttttgcatgggggctgacccaggccttgagcacctcttggcttcagcagccccctcccctgcaccacccacccctccaacctcactccgggccccgctgcccctgccagctgaccacaaggaaccctgggttgcaggtggcttccctgtccagggaggctccagctacccgttggggctgaccccctgcctataccggacgccaggaatgttcttctttgagtaaaggcagcctcgtctcgggcagtccctgcaggtccctcaccctccaggctgagcctggctctaggacctggagaactctcgaaggacccagccctggtgagccaaggacaaccacacagaaagccaggatggaaacggtgctcagcctggcccctggggcctctggacaaacttgggggtgaggggaagcggggcctcttgggattgactctgtggctctcagggccaataaagccatatgatgatga

SEQ ID NO: 6 - protein, 330 aa, Macaca mulatta
MQQQPLPGPGAPAAEPTKPPYSYIALIAMAIQSSPGQRATLSGIYRYIMGRFAFYRHNRPGWQNSIRHNLSLNECFVKVPRDDRKPGKGSYWTLDPDCHDMFEHGSFLRRRRRFTRRTGAEGTRGPAKARRGPLRATSQDPGVLDTTTSRQCSFPPELPDPKGLSFGGLVGAMPASMCPATTDGRPRPPTEPKEISTPKPACLGELPVATSSSSCPVFGFPAGFSEAGGFNKAPTPVSSPEASIGSSYQCRLQALNFCMGADPGLEHLLASAAPSPAPPTPPTSLRAPLPLPADHKEPWVAGGFPVQGGSSYPLGLTPCLYRTPGMFFFE

SEQ ID NO: 7 - DNA, 1336 bp, Bos taurus
cgtgcctggcctctgcggttccggcagcccggccagcccggccagcccggccagcatgcagcagccacccccacccgggcccctggcccctgcggccgagccaaccaagcctccttacagctacatcgccctgatcgccatggccatccagaactctccagggcagcgggccacactcagcggcatctaccgctacatcatgggccgcttcgccttctaccgccacaaccggccgggatggcagaacagtatccgccacaacctgtcactcaacgaatgctttgtcaaggtgccccgcgatgaccgcaagccgggcaagggcagctactggacgctggaccccgactgccatgacatgtttgagcatggcagctttctgcgccgccgccggcgcttcacccgacgggcaggtgctgagggcaccaagggccccaccaaagcgcgccgtggacccctccgagccaccagccaggacccaggagcccctgacgtcgcagctagcagacagtgcccattcccgccggagccactggaacccaagggcctaagctatgggggtctggtgggggccttgccagccagcatgtgcccggccaccaccaatgccaggcctcagacaccctcagaggccaaggagatgcccactcccaaggctacaggcccaggggagctccctgtggccacctcgtcttcctcgtgccctgcttttggctttcccaccagcttctctgaggctgaggggtttagcaaggcccctgcacccatcttgacccccgaggccaccatcgggagcagctaccagtgccggctgcaggcgctgaatttctgcatggggactgacccaggactggagcacctcttggcctcagcagccccctcccctgcaccatccacccctccagcctccctccgggccccgctgcccctgccagctgaccccaaggaaccctgggttgcaggcagcttccctgtccagggaagctccagctacccactggggctgaccccctgcctgtaccggacgccaggaatgttcttctttgagtgaaggccagccggcctcaggccgtccctgcatagcccctgccaactacgccctccaggttgagcctgactctgggatctggggagctctcaaaggatgcgggcctggcaagctcacgacagctgggacaggaagccaagattgcagcggtgaacactcagccagccctagggcctctggacagacttgggggtgaggggaagcagggccccctggggatttactctgtggctctcagggccaataaagccagtgtgatgatgaaaaaaaaaaaaaaaaaaaaaaaaaaaa

SEQ ID NO: 8 - protein, 330 aa, Bos taurus
MQQPPPPGPLAPAAEPTKPPYSYIALIAMAIQNSPGQRATLSGIYRYIMGRFAFYRHNRPGWQNSIRHNLSLNECFVKVPRDDRKPGKGSYWTLDPDCHDMFEHGSFLRRRRRFTRRAGAEGTKGPTKARRGPLRATSQDPGAPDVAASRQCPFPPEPLEPKGLSYGGLVGALPASMCPATTNARPQTPSEAKEMPTPKATGPGELPVATSSSSCPAFGFPTSFSEAEGFSKAPAPILTPEATIGSSYQCRLQALNFCMGTDPGLEHLLASAAPSPAPSTPPASLRAPLPLPADPKEPWVAGSFPVQGSSSYPLGLTPCLYRTPGMFFFE

SEQ ID NO: 9 - DNA, 1300 bp, Rattus norvegicus
cgcgcctggctctgcggtctcggcaacacagccagcatgcagcagccacccacagcagaatccttggctccttctacagagcccagcaagcccccttacagctacatagccctgattgccatggctatccagagttcaccgggtcagcgcgccaccctcagtggcatctaccgctacatcatgggccgctttgcattttaccggcacaaccggccaggctggcaaaacagcatccgccataacctgtctctcaacgagtgcttcgtcaaggtgcctcgtgatgaccgcaagccaggcaagggcagctactggaccctggatccagactgccacgacatgttccagcacggcagcttcctccgtcgccgcagacgcttcaccaagcgcacaggtgcccagggcaccaagggccctgtcaaggcagaccacagaccccttagagccaccagcccggatcaaggagcccccaacaccacaactggcagactgtgcccattcccaccagaggtgccaaaccccaagggctttgggggtctgatggggtcattgccagccaacatgtgcccaacaaccagtgacaccaggccccagctgcccactggacccaaagacatgtgctcggcaaagtctggaggtccaagggagctttctgaagccacctcgccctcaccatgcccggcattcggcttctcatctgccttctctgaagctgagagtctcggcaaggcccccacacccagtgtggctccggagagcatcgggagcagctaccagtgccggatgcagacgctgaatttctgtatgggggctgacccaggccttgaacacctcttgacctctgcagtcgccactcctggatcctcaacaccttcggcctctcaccgggctccactgcccctgccagctgactccaaggaaccgtgggtgcctgggggcttccctgtccagggaggttctggctatccactgggattacccccttgtctttaccggacaccgggaatgttcttcttcgagtgactgactgcagcagcccctctgtagggcttctgtcaactgttccctggagactcagactggctatgtgacctagggtattctgcaagaacccagccctggcaggccaaggactgtgaggaccggaaggcagaagtgagcagtcagccaggtccctggggggacctactgacaaacttgggatgtggggaaatggaaccctcaacatttttctctgtggttctcagggctaataaagccagtgagatgataaaaaaaaaaaaaaaaaaaaaaaaaaaaaagg

SEQ ID NO: 10 - protein, 327 aa, Rattus norvegicus
MQQPPTAESLAPSTEPSKPPYSYIALIAMAIQSSPGQRATLSGIYRYIMGRFAFYRHNRPGWQNSIRHNLSLNECFVKVPRDDRKPGKGSYWTLDPDCHDMFQHGSFLRRRRRFTKRTGAQGTKGPVKADHRPLRATSPDQGAPNTTTGRLCPFPPEVPNPKGFGGLMGSLPANMCPTTSDTRPQLPTGPKDMCSAKSGGPRELSEATSPSPCPAFGFSSAFSEAESLGKAPTPSVAPESIGSSYQCRMQTLNFCMGADPGLEHLLTSAVATPGSSTPSASHRAPLPLPADSKEPWVPGGFPVQGGSGYPLGLPPCLYRTPGMFFFE

SEQ ID NO: 11 - RNA, 21 nt, Artificial Sequence
  antisense oligonucleotide
caugugaugaugagggaaauu

SEQ ID NO: 12 - RNA, 21 nt, Artificial Sequence
  antisense oligonucleotide
uuguacacuacuacucccuuu

SEQ ID NO: 13 - RNA, 21 nt, Artificial Sequence
  antisense oligonucleotide
ggcucuaggaccugaagaauu

SEQ ID NO: 14 - RNA, 21 nt, Artificial Sequence
  antisense oligonucleotide
uuccgagauccuggacuucuu

SEQ ID NO: 15 - RNA, 21 nt, Artificial Sequence
  antisense oligonucleotide
ccaauaaagccaugugauguu

SEQ ID NO: 16 - RNA, 21 nt, Artificial Sequence
  antisense oligonucleotide
uugguuauuucgguacacuac

SEQ ID NO: 17 - DNA, 1722 bp, Homo sapiens
attcattgcgccgcggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

SEQ ID NO: 18 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 19 - DNA, 2112 bp, Homo sapiens
aaaacgggctcagttcgtaaaggagccgggtgacttcagaggcgccggcccgtccgtctgccgcacctgagcacggcccctgcccgagcctggcccgccgcgatgctgtagggaccgccgtgtcctcccgccggaccgttatccgcgccgggcgcccgccagacccgctggcaagatgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcacattgcatcttttcttcctaaaaaaatgcaaagatttacattgctgccaaatcatttcaactgaaaagaacagtattgctttgtaatagagtctgtaataggatttcccataggaagagatctgccagacgcgaactcaggtgccttaaaaagtattccaagtttactccattacatgtcggttgtctggttgccattgttgaactaaagcctttttttgattacctgtagtgctttaaagtatatttttaaaagggaggaaaaaaataacaagaacaaaacacaggagaatgtattaaaagtatttttgttttgttttgtttttgccaattaacagtatgtgccttgggggaggagggaaagattagctttgaacattcctggcgcatgctccattgtcttactattttaaaacattttaataatttttgaaaattaattaaagatgggaataagtgcaaaagaggattcttacaaattcattaatgtacttaaactatttcaaatgcataccacaaatgcaataatacaataccccttccaagtgcctttttaaattgtatagttgatgagtcaatgtaaatttgtgtttatttttatatgattgaatgagttctgtatgaaactgagatgttgtctatagctatgtctataaacaacctgaagacttgtgaaatcaatgtttcttttttaaaaaacaattttcaagttttttttacaataaacagttttgatttaaaatctcgtttgtatactattttcagagactttacttgcttcatgattagtaccaaaccactgtacaaagaattgtttgttaacaagaaaaaaa

SEQ ID NO: 20 - protein, 268 aa, Homo sapiens
MPRSFLVKKHFNASKKPNYSELDTHTVIISPYLYESYSMPVIPQPEILSSGAYSPITVWTTAAPFHAQLPNGLSPLSGYSSSLGRVSPPPPSDTSSKDHSGSESPISDEEERLQSKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 21 - DNA, 2112 bp, Homo sapiens; identical to SEQ ID NO: 19

SEQ ID NO: 22 - protein, 268 aa, Homo sapiens; identical to SEQ ID NO: 20

SEQ ID NO: 23 - DNA, 2219 bp, Homo sapiens
aaatctccctgagagcgggaccggcctcagctccaacacagcctccactgtgattaaaaataaaaattgctagagcagccctcactcgccacatctactttgatagctggctatttggaatttaaaggatatttgactttttctaacctcccatgaggctgtaagttccactgctccaaacccacccaccaaggactctgaacctgtccaccccgggcgcatcaagatcttccagctgggtacccccgatttgggccgactttgcacctccaaacaaccttagcatgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttgtgaagacctgtagaacctctttttgtgggtgatttttaaatatactgggctggacattccagttttagccaggcattggttaaaagagttagatgggatgatgctcagactcatctgatcaaagttccgagaggcatagaaggaaaaacgaagggccttagaggggcctacaaaccagcaacatgaaatggacaaaccaatctgcttaagatcctgtcatagttttagatcattggttatcctgatttgcaaagtgatcaaaagcattctagccatgtgcaaccaaacaccaccaaaaataaaatcaaacaaaactaagttgtgaaggaagggagggaaggtcatagccttcttaagcagaggtgttccattgttttagccaatccttggttgaatcttaggaatgaacagtgtctcaagctcattcacgtttcatgaccaactggtagttggcactgaaaaaacttttcagggctgtgtgaattgtgtgactgattgtcctagatgcactactttatttaaaaaataatgttcataaggagtcaatatgtagtttaagagacaatcagtgtgtgtcttataaatggtacatctgtggtttttaatctgtgctagacttcaaaactgtgatctcctgttattgtatgcaaccttgaactccacctctgcaggggttcttctgtgattaaataggttataattataagcaaaattcagagcaactgagtactgatctaaaaagattacctttggctggaggtgagctgcactgaaactttacgacaaaatgtctctggacaaagagagtcagagaagagaagcaaaaggacactaattcatctgtaatttactgttggtaagcctagcagtaaagagacattggtcaattgctctgaccctgatgaattattaaactgagatcattgtcgtttatgcttgcagatgttaaatggaaaagttatatatgcataaaccttttcttcctggatttggcagatatgtataattatattaaaatggttctagcacaaaaaaaaaaaaaaa

SEQ ID NO: 24 - protein, 297 aa, Homo sapiens
MMSYLKQPPYAVNGLSLTTSGMDLLHPSVGYPGPWASCPAATPRKQRRERTTFTRAQLDVLEALFAKTRYPDIFMREEVALKINLPESRVQVWFKNRRAKCRQQQQQQQNGGQNKVRPAKKKTSPAREVSSESGTSGQFTPPSSTSVPTIASSSAPVSIWSPASISPLSDPLSTSSSCMQRSYPMTYTQASGYSQGYAGSTSYFGGMDCGSYLTPMHHQLPGPGATLSPMGTNAVTSHLNQSPASLSTQGYGASSLGFNSTTDCLDYKDQTASWKLNFNADCLDYKDQTSSWKFQVL

SEQ ID NO: 25 - DNA, 2420 bp, Homo sapiens
cagggagtcccaccagcctagtcgccagaccttctgtgggatcatcggacccacctggaaccccacctgacccaagcccacctgctgcagcccactgcctggccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtgggcctctccagggccctgctgggctggaggcatggccagaggggtcatggccagcagctgcttgagacgaggatgcagtgtcaggaaaggaaggtctcacgggtagaaagcagccaggcgtggtggcgcacacctgtaatcccagctactcgggaggctgaggcaggagaatcgcttgaacccgggaggcggaggttgtggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaacctgcttcctaatggggatgcttcgccagccaggtcctcacctgtgtgtacaccagcaggacactgatccagtcacagccatacagctgtccacactgaagaacgtgtcctacaacagcctgaatcaaatggttagcttaatagataaaaatcccagactacttcagcctttaatgccttttattcataaaaactgtgaaagctagactgaaccattggaaacatttaactcagactctggattcagagtcgggaacccttagttctatctgaatccaagacagccacaccttagtatactgcccaaactaatgagtttaataaatacaaatactcgttaaaaaaaaaaaaaaaa

SEQ ID NO: 26 - protein, 435 aa, Homo sapiens [sequence omitted]

SEQ ID NO: 27 - DNA, 4815 bp, Homo sapiens
gtaaactccccgcgctggggcgggcggccgcgagccggcgagcgggcagagctcggcactgcgccggggcgcacggctcgggggacccaggcccagctaccttccctccgcccccgggctctgttctcactttccagcagtggaaggacgggaagcgggagccatgcagtccgaatcggggatcgtgccggatttcgaagtcggggaggagtttcatgaagagcccaaaacctattacgaactcaaaagtcaaccgctgaagagcagcagttccgccgagcatcctggggcctccaagcctccgataagctcctccagtatgacatcacgcatcttgctacgccagcaactcatgcgtgagcagatgcaggagcaggagcgcagggagcagcagcagaagctgcaggcggcccagttcatgcaacagagagtgcccgtgagtcagacaccagccataaacgtcagtgtgcccaccacccttccctctgccacgcaggtgccgatggaagtccttaaggtgcagacccacctcgaaaaccccaccaagtaccacatacagcaagcccaacggcagcaggtaaagcagtacctttctaccactttagcaaataaacatgccaaccaagtcctgagcttgccatgtccaaaccagcctggcgatcatgtcatgccaccggtgccggggagcagcgcacccaacagccccatggctatgcttacgcttaactccaactgtgaaaaagagggattttataagtttgaagagcaaaacagggcagagagcgagtgcccaggcatgaacacacattcacgagcgtcctgtatgcagatggatgatgtaatcgatgacatcattagcctagaatcaagttataatgaggaaatcttgggcttgatggatcctgctttgcaaatggcaaatacgttgcctgtctcgggaaacttgattgatctttatggaaaccaaggtctgcccccaccaggcctcaccatcagcaactcctgtccagccaaccttcccaacataaaaagggagctcacagagtctgaagcaagagcactggccaaagagaggcagaaaaaggacaatcacaacctgattgaacgaagaagaagatttaacataaatgaccgcattaaagaactaggtactttgattcccaagtcaaatgatccagacatgcgctggaacaagggaaccatcttaaaagcatccgtggactatatccgaaagttgcaacgagaacagcaacgcgcaaaagaacttgaaaaccgacagaagaaactggagcacgccaaccggcatttgttgctcagaatacaggaacttgaaatgcaggctcgagctcatggactttcccttattccatccacgggtctctgctctccagatttggtgaatcggatcatcaagcaagaacccgttcttgagaactgcagccaagacctccttcagcatcatgcagacctaacctgtacaacaactctcgatctcacggatggcaccatcaccttcaacaacaacctcggaactgggactgaggccaaccaagcctatagtgtccccacaaaaatgggatccaaactggaagacatcctgatggacgacaccctttctcccgtcggtgtcactgatccactcctttcctcagtgtcccccggagcttccaaaacaagcagccggaggagcagtatgagcatggaagagacggagcacacttgttagcgaatcctccctgcactgcattcgcacaaactgcttcctttcttgattcgtagatttaataacttacctgaaggggttttcttgataattttcctttaatatgaaattttttttcatgctttatcaatagcccaggatatattttatttttagaattttgtgaaacagacttgtatattctattttacaactacaaatgcctccaaagtattgtacaaataagtgtgcagtatctgtgaactgaattcaccacagactttagctttctgagcaagaggattttgcgtcagagaaatgtctgtccatttttattcaggggaaacttgatttgagatttttatgcctgtgacttccttggaaatcaaatgtaaagtttaattgaaagaatgtaaagcaaccaaaaagaaaaaaaaaaagaaagaaagaggaaaagaaatccatactaacccttttccattttataaatgtattgattcattggtactgccttaaagatacagtacccctctagctttgtttagtctttatactgcaaactatttaaagaaatatgtattctgtaaaagaaaaaaaaaatgcggccttttcatgaggatcgtctggttagaaaacataactgataccaaccgaaactgaagggagttagaccaaggctctgaaatataaagtctaatcttgctctcttttattctgtgctgttacagttttcttcatcaatgagtgtgatccagtttttcataagatattttattttgaaatggaaattaatgtcctctcaaagtaaaatattgaggagcactgaaagtatgttttactttttttttattttatttttgcttttgataagaaaaccgaactgggcatatttctaattggctttactatttttatttttaaattatgttttactgttcatttgatttgtacagattctttattatcattgttcttttcaatatatttgtattaatttgtaagaatatgcatcttaaaatggcaagttttccatatttttacaactcactggtggttttccgcattctttgtacacccatgaaagaaaacttttatgcaaggtcttgcatttaaaagacagctttgcgaatattttgtaaattacagtctcactcagaactgtttttggacacatttaaggtgtagtattaataggttaaaaccaggctttctagaaagaataaacttacatatttatttttaggacatgaaaatagcaatattcttggagattgataaccatagcattaatacgcccattatggtcatttaaattggggtttatttcagcaaacttgttgaatttatttttaagaaagaaatactgtattgggaagttactgttacttgataacaatgttttaacaagaagcaatgttataaagttagtttcagtgcattatctacttgtgtagtcctatgcaataacagtagtgttacatgtatcaagcctagatgttttatacagatgccatatagtgttatgagccaggctgttgaatggaatttctcagtagcagcctacaactgaatagcaagtggcataaagcatatccattcagaatgaagtgccttaaatatagcagtagtcttttttggactagcactgactgaactgtaatgtaggggaaagtttcatgatggtatctatagtcaagacgaacatgtagcatggtgcctatgtagacaatataagagcttccaattttccttcagatatttttaatattaaatatattttagtgacagagtgccaacttctttcatcaggaaaccttattcaggagggtttttaaaaagtgtttaaatgtcaaatgtgaattggtgatgggtgatggagggttcagagaggagtgatcgtcagatgtgtgaatggacggtttaggtgaaaataatcaactgcatagttcccatgcacgctgggcaatgagaatccttggaaacattggtgatgctatcagttttatagctttatttcttaagggggtagggaaaattagttcccattctttcaacccccttaactgtatagctcttttcctagaatagtgacgcaaatctgcatgaacagctaattgtaccatagtgttcattgatacaatcatagcattgtctatttttctcttcatatttatatgggggggagggcgctggatgcaaaagttgaagatcgtgatgctatgatgttagttttccttagctgattttgagggtttttaaaaataaagcaaggttgactaacctacggccacgggaacaggaccatggttaagcaaccatatagaaagctttgttgaaagaaagtatggcatcttgtaccactgccctgactgtcacaactcctaaccttgccattgcctgcctccccctccccttctccttaagagacaatttctgcaggtggcaggtgagcaagcccaggagaatgctgcaatcttgggggtggttttatttatttcttttttgccaaatagagtgtggattcatttcaggggctagctaagccaagaggcagtggtttgggcttgttgtttgtaacaagaaaatgatccacaccactcccccgattcccgggtgcagaattgtaactcggggttgggcctctatatggagtgaccaaaatgccaaaattgtccatctgcctctgagtagggcaatggaaataccaaaccttctgactttgccaaaaagcatacaagcaacctggtcatacataggatgacaaaattctttctggttgtttttaaacaataaagcaataagaacaaatacaatacataggaagttaaaagcacaaaggaatgaacttattaatatttttgaaaaatgcactgggaaaaagttgatgtcaataacagtataaaacagccctatttcttgataaaaaatgacaaatgactgtctcttgcggatgcttggtactgtaatgttaataatagtcacctgctgttggatgcagcaataatttctgtatggtccatagcactgtatattatggatcgatattaatgtatccaatgaaataatcgacttgttcttgatagcctcattaaagcatttggtttttcacatag

SEQ ID NO: 28 - protein, 520 aa, Homo sapiens
MQSESGIVPDFEVGEEFHEEPKTYYELKSQPLKSSSSAEHPGASKPPISSSSMTSRILLRQQLMREQMQEQERREQQQKLQAAQFMQQRVPVSQTPAINVSVPTTLPSATQVPMEVLKVQTHLENPTKYHIQQAQRQQVKQYLSTTLANKHANQVLSLPCPNQPGDHVMPPVPGSSAPNSPMAMLTLNSNCEKEGFYKFEEQNRAESECPGMNTHSRASCMQMDDVIDDIISLESSYNEEILGLMDPALQMANTLPVSGNLIDLYGNQGLPPPGLTISNSCPANLPNIKRELTESEARALAKERQKKDNHNLIERRRRFNINDRIKELGTLIPKSNDPDMRWNKGTILKASVDYIRKLQREQQRAKELENRQKKLEHANRHLLLRIQELEMQARAHGLSLIPSTGLCSPDLVNRIIKQEPVLENCSQDLLQHHADLTCTTTLDLTDGTITFNNNLGTGTEANQAYSVPTKMGSKLEDILMDDTLSPVGVTDPLLSSVSPGASKTSSRRSSMSMEETEHTC

SEQ ID NO: 29 - DNA, 1752 bp, Homo sapiens
gagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacctggcagggaacaaccaagactggggttaaatctcacagcctgcaagtggaagagaagaacttgaacccaggtccaacttttgcgccacagcaggctgcctcttggtcctgacaggaagtcacaacttggccctgacttcctatcctagggaaggggccggctggagaggccaggacagagaaagcagatcccttctttttccaaggactctgtgtcttccataggcaacatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaaaacatctcctgccagctgaactgtagttagaatctctgggcctctcctcaactgtcctggacccaaggctaggaaagggctgcttgagatgactgtggtccccccttagactccctaagcccgagtgagctcaggtgtcaccctgttctcaagttgggggatgggtaataaaggagggggaattcccttgaacaagaagaactggggatagttatatttccacctgcccttgaagctttaagacagtgatttttgtgtaaggttgtatttcaaagactcgaattcattttctcagtcatttcctttgtaacagagttttacgacttagagtctgtgaaaacaggcaaggagcccgggttaaaatatccccctattcgcccccaaaatgcaataaaagaagataaaagagagaggata

SEQ ID NO: 30 - protein, 317 aa, Homo sapiens
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDLRKMVDMLQDSFPARFKAIHFIHQPWYFTTTYNVVKPFLKSKLLERVFVHGDDLSGFYQEIDENILPSDFGGTLPKYDGKAVAEQLFGPQAQAENTAF

SEQ ID NO: 31 - DNA, 25 bp, Artificial Sequence
  inhibitory RNA
tccctacagctacatcgcccttatt

SEQ ID NO: 32 - DNA, 25 bp, Artificial Sequence
  inhibitory RNA
ccacccatggagcccaaagagattt

SEQ ID NO: 33 - DNA, 25 bp, Artificial Sequence
  inhibitory RNA
cggacgccaggaatgttcttctttg

SEQ ID NO: 34 - DNA, 57 bp, Artificial Sequence
  synthetic oligonucleotide
gccaggaatgttcttctttgttcaagagacaaagaagaacattcctggcttttttgt

SEQ ID NO: 35 - DNA, 61 bp, Artificial Sequence
  synthetic oligonucleotide
ctagacaaaaaagccaggaatgttcttctttgtctcttgaacaaagaagaacattcctggc

SEQ ID NO: 36 - DNA, 55 bp, Artificial Sequence
  synthetic oligonucleotide
gccaataaagccatgtgatttcaagagaatcacatggctttattggcttttttgt

SEQ ID NO: 37 - DNA, 59 bp, Artificial Sequence
  synthetic oligonucleotide
ctagacaaaaaagccaataaagccatgtgattctcttgaaatcacatggctttattggc

SEQ ID NO: 38 - DNA, 55 bp, Artificial Sequence
  synthetic oligonucleotide
gcatctaccgctacatcatttcaagagaatgatgtagcggtagatgcttttttgt

SEQ ID NO: 39 - DNA, 59 bp, Artificial Sequence
  synthetic oligonucleotide
ctagacaaaaaagcatctaccgctacatcattctcttgaaatgatgtagcggtagatgc

SEQ ID NO: 40 - DNA, 22 bp, Artificial Sequence
  synthetic oligonucleotide
ttgtccatctgcctctgagtag

SEQ ID NO: 41 - DNA, 23 bp, Artificial Sequence
  synthetic oligonucleotide
cctatgtatgaccaggttgcttg

SEQ ID NO: 42 - DNA, 24 bp, Artificial Sequence
  synthetic oligonucleotide
tggtgtagttctgagtgtggtggt

SEQ ID NO: 43 - DNA, 26 bp, Artificial Sequence
  synthetic oligonucleotide
agtccatgaaaggtgacagggatgtt

SEQ ID NO: 44 - DNA, 25 bp, Artificial Sequence
  synthetic oligonucleotide
tgtcagatgaggacagtgggaaagg

SEQ ID NO: 45 - DNA, 25 bp, Artificial Sequence
  synthetic oligonucleotide
ctgaagtagaggagaaggacgaagg

SEQ ID NO: 46 - DNA, 20 bp, Artificial Sequence
  synthetic oligonucleotide
agcgaactggacacacatac

SEQ ID NO: 47 - DNA, 19 bp, Artificial Sequence
  synthetic oligonucleotide
tctagactgggcatcgcag

SEQ ID NO: 48 - DNA, 20 bp, Artificial Sequence
  synthetic oligonucleotide
gtccgcagtcttagcaggag

SEQ ID NO: 49 - DNA, 22 bp, Artificial Sequence
  synthetic oligonucleotide
gcttgagggtctgaatcttgct

SEQ ID NO: 50 - DNA, 20 bp, Artificial Sequence
  synthetic oligonucleotide
gccaggaatgttcttctttg

SEQ ID NO: 51 - DNA, 19 bp, Artificial Sequence
  synthetic oligonucleotide
gccaataaagccatgtgat

SEQ ID NO: 52 - DNA, 19 bp, Artificial Sequence
  synthetic oligonucleotide
gcatctaccgctacatcat

(19) United States
(12) Patent Application Publication
(10) Pub. No.: US 2017/0082611 A1
(43) Pub. Date: Mar. 23, 2017

(54) Methods for Inhibiting Epithelial to Mesenchymal Transition by Inhibition of FOXS1

(71) Applicant: Regenerative Research Foundation, Rensselaer, NY (US)

(72) Inventors: Jeffrey Stern, Slingerlands, NY (US); Sally Temple Stern, Slingerlands, NY (US); Timothy Blenkinsop, Rensselaer, NY (US)

(21) Appl. No.: 15/272,088

(22) Filed: Sep. 21, 2016

Related U.S. Application Data
(60) Provisional application No. 62/221,500, filed on Sep. 21, 2015.

(51) Int. Cl. G01N 33/50; C12N 15/113; A61K 31/455; C12Q 1/68; A61K 31/4439

(52) U.S. Cl. CPC G01N 33/5041; C12Q 1/6886; C12Q 1/6883; G01N 33/5023; G01N 33/5044; A61K 31/4439; A61K 31/455; C12N 15/113; C12Q 2600/136; C12Q 2600/158; C12N 2310/11; C12N 2320/30

(57) ABSTRACT

Methods for inhibiting epithelial to mesenchymal transition (EMT) in epithelial cells are disclosed. The methods can include contacting epithelial cells, such as retinal pigment epithelial cells or breast epithelial cells, with an inhibitor of the forkhead box s1 (FOXS1) signaling pathway.

### CROSS-REFERENCE TO RELATED APPLICATIONS

This application claims the benefit of U.S. Application No. 62/221,500, filed on Sep. 21, 2015, which is incorporated by reference herein in its entirety.

### TECHNICAL FIELD

This invention relates to the field of cell biology, and more particularly to methods for inhibiting epithelial to mesenchymal transition (EMT).

### BACKGROUND

The healthy retina is a smooth film that coats the back of the eye and mediates vision. In pathologic conditions, epiretinal membranes (ERMs) grow on the retinal surface, distorting retinal anatomy resulting in vision loss. ERMs arise from cells displaced onto the inner retinal surface that proliferate to form fibrous, contractile membranes. The most common type of displaced cell is the retinal pigment epithelial (RPE) cell. RPE cells occur normally in a cobblestone epithelia located under the retina known as the RPE layer. In the healthy RPE layer, RPE cells are attached to a thick basement membrane (Bruchs membrane).

In disease, RPE cells detach from Bruchs membrane and then proliferate on the inner retinal surface to form ERM. The displaced RPE cells can also proliferate under the retina and cause subretinal membranes. The pathophysiology of ERM formation after displacement of RPE cells from their niche on Bruchs membrane involves epithelial to mesenchymal transition (EMT). After RPE cells undergo EMT, they proliferate to form myocontractile fibrous ERM. Mild ERMs in the macular region, known as macular pucker, are most prevalent, causing moderate vision loss that is addressed by surgical peeling. Extensive ERM growth, known as proliferative vitreoretinopathy (PVR), can also occur. PVR is the most common cause of irreparable retinal detachments and blind painful eyes in developed countries.

Current drug therapies for diseases associated with EMT are lacking, and although surgery is currently the best option for macular pucker, significant surgical morbidity and a high rate of disease recurrence limits success. Surgical repair of PVR is yet more invasive and less successful than surgery for macular pucker.

Novel and improved therapies for inhibiting EMT formation and for treating diseases and conditions associated with EMT are needed in the art.

### SUMMARY

As follows from the Background section, above, there is a need in the art for novel and improved therapies for inhibiting EMT and for treating diseases and conditions associated with EMT. The present disclosure provides these and other, related advantages.

Thus, in certain aspects, the present disclosure provides a method for inhibiting epithelial to mesenchymal transition (EMT) in epithelial cells. The method can include contacting the epithelial cells with an inhibitor of the forkhead box s1 (FOXS1) signaling pathway. In some aspects, the inhibitor is a molecule such as an antisense oligonucleotide, a small molecule, a peptide, and a ribozyme. In some aspects, the inhibitor is an antisense such as a double-stranded RNA (d5RNA) molecule or analogue thereof, a double-stranded DNA (dsDNA) molecule or analogue thereof, a short hairpin RNA molecule, or a small interfering RNA (siRNA) molecule. In some aspects, the inhibitor is an siRNA or shRNA molecule comprising or consisting of a sequence set forth in one of SEQ ID NOs. 31-33, 34-39 and 50-52. In some aspects, the inhibitor is an inhibitor of human FOXS1. In some aspects, the inhibitor targets a member of the p38 signaling pathway. In some aspects, the inhibitor is the small molecule p38 inhibitor SB202190. In some aspects, the inhibitor is nicotinamide. In some aspects, the epithelial cells are retinal pigment epithelial (RPE) cells. In some aspects, the epithelial cells are breast epithelial cells. In some aspects, the epithelial cells are RPE cells in a subject. In some aspects, the epithelial cells are cultured RPE cells. In some aspects, the epithelial cells are breast epithelial cells in a subject. In some aspects, the epithelial cells are cultured breast epithelial cells.

Also provided herein is a method for treating a disease or disorder associated with EMT. The method can include administering to a subject in need of such treatment a composition containing an inhibitor of the FOXS1 signaling pathway. Typically, the inhibitor is present in an effective amount for decreasing FOXS 1 expression in the subject. In some aspects, the disease or disorder is epiretinal membrane formation (ERM), proliferative vitreoretinopathy (PVR), or macular pucker. In some aspects, the disease or disorder is abnormal breast epithelial cell growth. In some aspects, the abnormal breast epithelial cell growth is breast cancer. In some aspects, the inhibitor is present in an amount effective for decreasing the expression of one or more of SNAIL, SLUG, and TWIST in the subject. In some aspects, the method includes measuring the expression level of FOXS1 in the subject. In some aspects, the expression level of FOXS1 is measured in a surgically removed tissue affected by EMT. In some aspects, the method further includes measuring the expression level in the subject of one or more of SNAIL, SLUG, and TWIST. In some aspects, the expression level of the one or more of SNAIL, SLUG, and TWIST is measured in a surgically removed tissue sample affected by EMT. In some aspects, the inhibitor is present in an amount effective for increasing the expression of one or both of OTX2 and Bestrophin in the subject. In some aspects, the method further includes measuring the expression level in the subject of one or both of OTX2 and Bestrophin. In some aspects, the expression level of one or both of OTX2 and Bestrophin is measured in a surgically removed retinal tissue sample affected by EMT.

In any of the above methods for treating a disease or disorder associated with EMT, the inhibitor can be an antisense oligonucleotide, a small molecule, a peptide, or a ribozyme. In some aspects, the inhibitor is double-stranded RNA (d5RNA) molecule or analogue thereof, a double-stranded DNA (dsDNA) molecule or analogue thereof, a short hairpin RNA molecule, or a small interfering RNA (siRNA) molecule. In some aspects, the inhibitor is an inhibitor of human FOXS1. In some aspects, the inhibitor targets a member of the p38 signaling pathway. In some aspects, the inhibitor is the small molecule p38 inhibitor SB202190. In some aspects, the inhibitor is an siRNA or shRNA molecule comprising or consisting of a sequence set forth in one of SEQ ID NOs. 31-33, 34-39 and 50-52. In some aspects, the inhibitor is nicotinamide. In some aspects, the epithelial cells are RPE cells.

Also provided herein is a method of screening for a compound that inhibits EMT in epithelial cells. The method can include: providing a monolayer of epithelial cells; culturing the monolayer of cells in conditions that induce the cells to undergo EMT; contacting the monolayer of cells with a test compound; determining the expression level of at least one member of the FOXS1 signaling pathway; and identifying the test compound as a candidate inhibitor of EMT if the expression level of the at least one member of the FOXS1 signaling pathway is decreased relative to a control or a reference level. Further provided herein is a method of screening for a compound that inhibits EMT in epithelial cells. The method can include: providing a monolayer of epithelial cells; culturing the monolayer of cells in conditions that induce the cells to undergo EMT; contacting the monolayer of cells with a test compound; determining the expression level of one or more of the EMT-associated markers selected from the group consisting of FOXS1, SLUG, SNAIL, and TWIST; and identifying the test compound as a candidate inhibitor of EMT if the expression level of the one or more markers is decreased relative to a control or reference level. In some aspects, the epithelial cells are RPE cells. In some aspects, the epithelial cells are breast epithelial cells. In some aspects, the method further includes measuring the expression level of one or both of OTX2 and Bestrophin. In some aspects, the at least one member of the FOXS1 signaling pathway is FOXS1 or p38. In some aspects, culturing the cells under conditions that induce EMT includes contacting the cells with one or both of TNFα and TGFβ. In some aspects, the expression level is gene expression level. In some aspects, the gene expression level is measured using quantitative real-time polymerase chain reaction (PCR). In some aspects, the expression level is protein expression level. In some aspects, the protein expression level is determined using an assay such as immunoblot, immunohistochemistry, fluorescence microscopy, ELISA, or multiplex assay.

Also provided herein is a pharmaceutical formulation containing: (a) an effective amount for inhibiting the FOXS1 signaling pathway of an inhibitor such as an antisense oligonucleotide, a small molecule, a peptide, or a ribozyme, and (b) a pharmaceutical carrier; wherein the inhibitor reduces FOXS1 expression level and/or activity when administered to a subject suffering from EMT. In some aspects of the formulation, the formulation is for use in the treatment of a disease or disorder associated with EMT. In some aspects, the disease or disorder is ERM formation, macular pucker, or PVR. In some aspects, the disease or disorder is abnormal breast epithelial cell growth. In some aspects, the abnormal breast epithelial cell growth is breast cancer. In some aspects, the inhibitor is an inhibitor of FOXS1. In some aspects, the inhibitor is an inhibitor of the p38 signaling pathway. In some aspects, the inhibitor inhibits p38. In some aspects, the inhibitor is the small molecule p38 inhibitor SB202190. In some aspects, the inhibitor is an siRNA or shRNA molecule comprising or consisting of a sequence set forth in one of SEQ ID NOs. 31-33, 34-39 and 50-52. In some aspects, the inhibitor is nicotinamide.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 contains photographs of RPE cells cultured in the indicated conditions with a magnification of approximately 20×.

FIG. 2 contains bar graphs quantifying transcript level for the indicated transcription factors in RPE cells treated according to the indicated conditions over a time course (day “D” 1 to 10).

FIG. 3 contains photographs of microscopic images of RPE cells cultured in the indicated conditions for 4 days and then fixed and immunostained for p38; magnification about 40×.

FIG. 4, left panel, contains bar graphs of the transcript levels of SNAIL, SLUG and TWIST in RPE cells cultured with the indicated conditions (+/−TFGβ, TNFα and p38 inhibitor). The right panel contains photographs of the RPE cells treated with the indicated conditions.

FIG. 5 contains bar graphs quantifying transcript levels of FOXS1 (upper panel), SNAIL (middle panel), and SLUG (lower panel) in RPE cells cultured in the indicated conditions. FOXS1 a-c correspond to three different knockdown constructs for FOXS1. “Pb”: polybrene; “SV” indicates scrambled vector construct (control).

FIG. 6 is a bar graph quantifying FOXS1 levels in RPE cells cultured in the indicated conditions; “TnT” corresponds to TNFα+TGFβ; p38i: p38 inhibitor.

FIG. 7 contains bar graphs quantifying transcript levels of SNAIL (left panel), SLUG (middle panel) and TWIST (right panel) in RPE cells that overexpress FOXS1, compared to a control or RPE cells transfected with a scrambled virus (control). Error bars indicate standard error of the mean (SEM).

FIG. 8 contains bar graphs quantifying transcript expression levels of SNAIL, SLUG, TWIST, and FOXS1 in hTERT HMEnt breast epithelial cells cultured in the indicated conditions.

FIG. 9 contains bar graphs quantifying transcript levels of SNAIL, SLUG, FOXS1, MITF, OTX2 and Bestrophin (“BEST”) in epiretinal membranes taken from living human vitreous from two patients (“JS PVR1” and “JS PVR2”) compared to normal RPE cells cultured in vitro (“RPE”) and RPE cells cultured in EMT-inducing conditions (RPE-EMT).

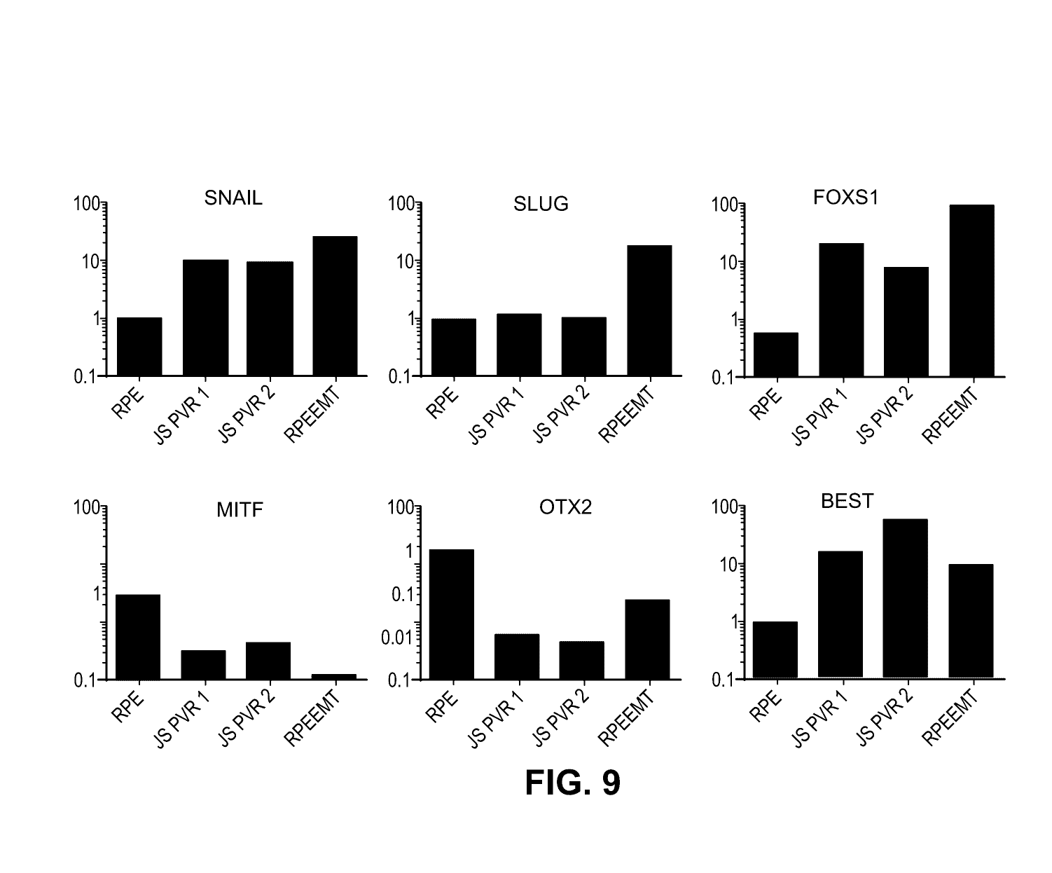

FIG. 10 is a bar graph quantifying transcript levels of SNAIL, FOXS1, RPE65 and BEST1 in RPE cells cultured in the indicated conditions. Error bars indicate standard error of the mean (SEM).

FIG. 11 is a bar graph quantifying transcript levels of SNAIL, SLUG, and FOXS1 in RPE cells cultured in the indicated conditions. Error bars indicate standard error of the mean (SEM).

### DETAILED DESCRIPTION

### Overview

The present disclosure is based, at least in part, on the discovery that FOXS1 signaling drives epithelial to mesenchymal transition (EMT), which leads to ERM formation in epithelial cells. FOXS1 signaling may arise via the p38, or other, upstream signaling pathways. Thus, provided herein are methods for inhibiting EMT, and for treating diseases and disorders associated with EMT. In a specific embodiment, the methods include inhibiting the transcription factor FOXS1.

The present Examples, below, describe a novel in vitro culture method for screening agents that inhibit EMT. As described in Example 1, RPESCs were used to generate RPE monolayers which exhibit characteristic RPE appearance. Further, a combination of TNFα and TFGβ induced normal, healthy RPE cells to undergo EMT (Example 2). It was also demonstrated that RPE cells undergoing EMT up-regulate the transcription factors SNAIL and SLUG (Example 3), and, further, that the p38 signaling pathway can drive EMT, since inhibition of p38 blocked up-regulation of EMT-associated transcription factors (Example 4). It was also demonstrated that EMT is mediated by activation of FOXS1 (Example 5), which is downstream of p38 (Example 6). It was further demonstrated that FOXS1 is sufficient for EMT induction in RPE (Example 7), and also is upregulated in breast epithelium (Example 8). Moreover, the Examples demonstrate that pathways underlying EMT in the in vitro RPE model of EMT are also active in ERMs surgically removed from patients (Example 9). As described in Example 10, FOXS1 can be inhibited to treat patients suffering from a disease or disorder associated with EMT.

### Definitions

As used herein, the term “subject” means any animal, including any vertebrate, including any mammal, and, in particular, a human, and can also be referred to, e.g., as an individual or patient. A non-human mammal can be, for example, without limitation a non-human primate (such as a monkey, baboon, gorilla, or orangutan), a bovine animal, a horse, a whale, a dolphin, a sheep, a goat, a pig, a dog, a feline animal (such as a cat), a rabbit, a guinea pig, a hamster, a gerbil, a rat, or a mouse. Non-mammalian vertebrates include without limitation, a bird, a reptile, or a fish.

As used herein, the terms “reducing” and “inhibiting” are interchangeable, and mean any level of reduction up to and including complete inhibition (e.g., of an expression level and/or activity of a target gene or gene product (e.g., mRNA or polypeptide).

As used herein, “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; and/or (2) inhibiting the state, disorder or condition, i.e., arresting, reducing or delaying the development of the disease or a relapse thereof (in case of maintenance treatment) or at least one clinical or sub-clinical symptom thereof; and/or (3) relieving the disease, i.e., causing regression of the state, disorder or condition or at least one of its clinical or sub-clinical symptoms; and/or (4) causing a decrease in the severity of one or more symptoms of the disease. The benefit to a subject to be treated is either statistically significant or at least perceptible to the patient or to the physician.

A “disease or disorder associated with EMT” can include, but is not limited to, a disease associated with ERM formation, abnormal epithelial cell proliferation in, e.g., breast, lung, bowel, and other epithelial cancers, as well as EMT-mediated fibrotic diseases known to occur in the heart, lung, breast, kidney and intestine.

As used herein, “treating a disease or disorder associated with EMT” means inhibiting further EMT. The term can also include causing cells that have undergone EMT to revert to their normal epithelial phenotype (e.g., mesenchymal to endothelial transition or “MET”).

As used herein, the term “preventing a disease” (e.g., a disease or disorder associated with EMT) in a subject means for example, to inhibit or stop the development of one or more symptoms of a disease in a subject before they occur or are detectable, e.g., by the patient or the patient's doctor. Preferably, the disease or disorder (e.g., ERM formation, macular pucker, PVR, breast cancer, cardiac fibrosis, etc., as described herein) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

As used herein “combination therapy” means the treatment of a subject in need of treatment with a certain composition or drug in which the subject is treated or given one or more other compositions or drugs for the disease in conjunction with the first and/or in conjunction with one or more other therapies, such as, e.g., surgery or other therapeutic intervention. Such combination therapy can be sequential therapy wherein the patient is treated first with one treatment modality, and then the other, and so on, or all drugs and/or therapies can be administered simultaneously. In either case, these drugs and/or therapies are said to be “coadministered.” It is to be understood that “coadministered” does not necessarily mean that the drugs and/or therapies are administered in a combined form (i.e., they may be administered separately or together to the same or different sites at the same or different times). For example, an inhibitor of the FOXS1 pathway may be coadministered with surgery and/or a steroid and/or an antimetabolite drug and/or a chemotherapeutic agent (e.g. to treat cancer, e.g., breast cancer).

As used herein, the term “pharmaceutically acceptable” refers to molecular entities and compositions that are generally believed to be physiologically tolerable and do not typically produce an allergic or similar untoward reaction, such as gastric upset, dizziness and the like, when administered to a human.

The term “pharmaceutically acceptable derivative” as used herein means any pharmaceutically acceptable salt, solvate or pro drug, e.g., ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol. 1: Principles and Practice, which is incorporated herein by reference to the extent of teaching such derivatives. Pharmaceutically acceptable derivatives include salts, solvates, esters, carbamates, and/or phosphate esters.

As used herein the terms “therapeutically effective” and “effective amount,” used interchangeably, applied to a dose or amount refer to a quantity of a composition, compound or pharmaceutical formulation that is sufficient to result in a desired activity upon administration to an animal in need thereof. Within the context of the present invention, the term “therapeutically effective” refers to that quantity of a composition, compound or pharmaceutical formulation that is sufficient to reduce or eliminate at least one symptom of a disease or condition specified herein, e.g., cancer, anemia, iron overload, etc. When a combination of active ingredients is administered, the effective amount of the combination may or may not include amounts of each ingredient that would have been effective if administered individually. The dosage of the therapeutic formulation will vary, depending upon the nature of the disease or condition, the patient's medical history, the frequency of administration, the manner of administration, the clearance of the agent from the host, and the like. The initial dose may be larger, followed by smaller maintenance doses. The dose may be administered, e.g., weekly, biweekly, daily, semi-weekly, etc., to maintain an effective dosage level.

The term “nucleic acid hybridization” refers to the pairing of complementary strands of nucleic acids. The mechanism of pairing involves hydrogen bonding, which may be Watson-Crick, Hoogsteen or reversed Hoogsteen hydrogen bonding, between complementary nucleoside or nucleotide bases (nucleobases) of the strands of nucleic acids. For example, adenine and thymine are complementary nucleobases that pair through the formation of hydrogen bonds. Hybridization can occur under varying circumstances. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3rd ed., New York and London: Garland Publ., 1994, Ch. 7.

Typically, hybridization of two strands at high stringency requires that the sequences exhibit a high degree of complementarity over an extended portion of their length. Examples of high stringency conditions include: hybridization to filter-bound DNA in 0.5 M NaHPO4, 7% SDS, 1 mM EDTA at 65° C., followed by washing in 0.1×SSC/0.1% SDS (where 1×SSC is 0.15 M NaCl, 0.15 M Na citrate) at 68° C. or for oligonucleotide (oligo) inhibitors washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos).

Conditions of intermediate or moderate stringency (such as, for example, an aqueous solution of 2×SSC at 65° C.; alternatively, for example, hybridization to filter-bound DNA in 0.5 M NaHPO4, 7% SDS, 1 mM EDTA at 65° C. followed by washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA or RNA molecule and length and base composition of the probe, and are normally determined empirically in preliminary experiments, which are routine (see Southern, J. Mol. Biol. 1975; 98:503; Sambrook et al., Molecular Cloning: A Laboratory Manual, 2nd ed., vol. 2, ch. 9.50, CSH Laboratory Press, 1989; Ausubel et al. (eds.), 1989, Current Protocols in Molecular Biology, Vol. I, Green Publishing Associates, Inc., and John Wiley & Sons, Inc., New York, at p. 2.10.3). An extensive guide to the hybridization of nucleic acids is found in, e.g., Tijssen (1993) Laboratory Techniques in Biochemistry and Molecular Biology—Hybridization with Nucleic Acid Probes part I, chapt 2, “Overview of principles of hybridization and the strategy of nucleic acid probe assays,” Elsevier, N.Y. (“Tijssen”).

As used herein, the term “standard hybridization conditions” refers to hybridization conditions that allow hybridization of two nucleotide molecules having at least 50% sequence identity. According to a specific embodiment, hybridization conditions of higher stringency may be used to allow hybridization of only sequences having at least 75% sequence identity, at least 80% sequence identity, at least 85% sequence identity, at least 90% sequence identity, at least 95% sequence identity, or at least 99% sequence identity.

As used herein, the phrase “under hybridization conditions” means under conditions that facilitate specific hybridization of a nucleic acid sequence to a complementary sequence. The terms “hybridizing specifically to” and “specific hybridization” and “selectively hybridize to,” as used herein refer to the binding, duplexing, or hybridizing of a nucleic acid molecule preferentially to a particular nucleotide sequence under at least moderately stringent conditions, and preferably, highly stringent conditions, as discussed above.

“Polypeptide” and “protein” are used interchangeably and mean any peptide-linked chain of amino acids, regardless of length or post-translational modification.

As used herein, the term “nucleic acid” or “oligonucleotide” refers to a deoxyribonucleotide or ribonucleotide in either single- or double-stranded form. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev 6:153-156). The term nucleic acid is used interchangeably with cDNA, cRNA, mRNA, oligonucleotide, probe and amplification product.

### Forkhead Box S1 (FOXS1) Signaling Pathway and Inhibitors Thereof

It is presently discovered that FOXS1 is necessary and sufficient for driving EMT in epithelial cells, such as, but not limited to, RPE and breast epithelium. FOXS1 has been previously identified in peripheral sensory neurons (see, Montelius et al., Differentiation. 2007 June; 75(5):404-17), but no role for FOXS1 in EMT induction has been previously described.
Human FOXS1 has the mRNA nucleic acid sequence set forth in GenBank® Accession No. NM_004118 (SEQ ID NO: 1), and the amino acid sequence set forth in GenBank® Accession No. NP_004109 (SEQ ID NO: 2). In certain embodiments, also encompassed herein are mammalian orthologs of human FOXS1. By way of non-limiting examples, the GenBank® Accession Numbers for the nucleic and amino acid sequences of exemplary mammalian FOXS 1 sequences are provided in Table 1, below:

TABLE 1
FOXS1 Orthologs

| | SEQ | | SEQ | |
|---|---|---|---|---|
| | ID | GENBANK NO. | ID | GENBANK NO. |
| FOXS1 species | NO: | (nucleic acid) | NO: | (protein) |
| Mus musculus | 3 | NM_010226.2 | 4 | NP_034356 |
| Macaca mulatta | 5 | NM_001190859 | 6 | NP_001177788 |
| Bos taurus | 7 | NM_001099716.1 | 8 | NP_001093186 |
| Rattus norvegicus | 9 | NM_001012091 | 10 | NP_001012091 |

It is presently discovered that the p38 signaling pathway induces FOXS1 transcription. As demonstrated in the present Examples, below, p38 can be targeted by an inhibitor to decrease FOXS1 expression, which leads to inhibition of EMT.

The skilled artisan will appreciate that other mediators of the p38 signaling pathway can be targeted according to the present methods, since those mediators are known. Such mediators can be upstream of p38, e.g., MAP kinase, and many others, or downstream. See, for example, Banerjee A et al. Curr Opin Pharmacol. 2012 June; 12(3):287-92; Yong H Y, et al. Expert Opin Investig Drugs. 2009 December; 18(12):1893-905; Kirkwood K L and Rossa C Jr. Curr Drug Metab. 2009 January; 10(1):55-67; Clark J E, et al. Pharmacol Ther. 2007 November; 116(2):192-206. Any p38 signaling pathway member can be targeted according to the present methods, so long as inhibition of the targeted member causes a decrease in FOXS1 expression and/or activity (i.e., induction of EMT in epithelial cells).

Non-limiting examples of known p38 inhibitors that can be used in the present methods include, but are not limited to, e.g., AMG548 (p38 MAPK inhibitor), AS1940477 (p38 MAP kinase inhibitor), CBS3830 (p38 MAPK inhibitor), Dilmapimod SB-6813123 (p38 MAP kinase inhibitor), Doramapimod|BIRB-796 (p38 MAPK inhibitor), FR-167653 (p38 MAPK inhibitor), JLU1124 (p38 MAPK inhibitor), LASSBio-998 (p38 MAPK inhibitor), Losmapimod (GW856553) (p38 MAP kinase inhibitor), LY2228820 (p38 MAP kinase inhibitor), LY3007113 (p38 MAP kinase inhibitor), ML3403 (p38 MAP kinase inhibitor), Pamapimod (p38 MAP kinase inhibitor), PD-98059|PD098059 (p38 MAP kinase inhibitor), PD-169316 (p38 MAP kinase inhibitor), PH-797804 (p38 MAP kinase inhibitor), R-130823 (p38 MAP kinase inhibitor), R03201195 (p38 MAP kinase inhibitor), RPR-200765A (p38 MAP kinase inhibitor), RPR-203494(p38 MAP kinase inhibitor), RWJ-67657 (p38 MAP kinase inhibitor), SB-203580 (p38 MAP kinase inhibitor), SB-239063 (p38 MAP kinase inhibitor), SB-242235 (p38 MAPK inhibitor), SCIO-323 (p38 MAP kinase inhibitor), SD-282 (p38 MAPK inhibitor), Semapimod|CNI-1493 (p38 MAPK inhibitor), Soblidotin|TZT-1027 (p38 MAPK inhibitor), TAK-715 (p38 MAPK inhibitor), Talmapimod|SCIO-469 (p38 MAPK inhibitor), UO126 (p38 MAPK inhibitor), UR-13756 (p38 MAPK inhibitor), VX-702 (p38 MAPK inhibitor), and VX-745 (p38 MAPK inhibitor).

The skilled artisan will readily appreciate that other inhibitors of the p38 signaling pathway are also contemplated for use in the present methods. Methods for identifying suitable inhibitors of p38 signaling that cause a decrease in FOXS1 expression and/or activity are described in detail, below.

Other pathways that regulate FOXS1 expression and/or activity can also be targeted according to the present methods. For signaling pathways that cause increases in FOXS1 expression, it is presently contemplated to administer antagonists of those pathways, in order to inhibit and/or decrease FOXS1 expression.

For signaling pathways that cause decreases in FOXS1 expression and/or activity, it is presently contemplated that agonists of those pathways can be used to increase those pathways, in order to induce decreases in FOXS1 expression and/or activity. Decreases in FOXS1 expression and/or activity can be determined according to any suitable method known in the art (e.g., for expression, FOXS1 transcripts or protein can be detected, and for activity, the induction of EMT-associated transcripts, shown herein to be regulated by FOXS1 (e.g., SNAIL, SLUG, TWIST) can be detected (by PCR, Western blot and the like). Preferably, according to the methods disclosed herein, the change in expression and/or activity is a statistically significant change, and, e.g., an at least 1.5-fold, 2-fold, 3-fold, 4-fold, 5-fold, 10-fold or more change.

In other embodiments, FOXS1 expression and/or activity can be inhibited by directly targeting FOXS1. Methods for inhibiting transcription and/or translation and/or activity of target transcription factors are known in the art. Non-limiting examples are provided below.

In some embodiments, an inhibitor of FOXS1 can be an antisense oligonucleotide, a small molecule, a peptide, or a ribozyme. Because the nucleic acid sequence of FOXS1 is known, it is within the skill in the art to design various antisense oligonucleotides that specifically target (i.e., specifically hybridize to) FOXS1.

Examples of antisense oligonucleotides include, for example, and without limitation, double-stranded RNA (d5RNA) molecules or analogues thereof, double-stranded DNA (dsDNA) molecules or analogues thereof, short hairpin RNA molecules, and small interfering RNA (siRNA) molecules.

Antisense Nucleic Acids

By way of example, antisense oligonucleotides typically are about 5 nucleotides to about 30 nucleotides in length, about 10 to about 25 nucleotides in length, or about 20 to about 25 nucleotides in length. For a general discussion of antisense technology, see, e.g., Antisense DNA and RNA, (Cold Spring Harbor Laboratory, D. Melton, ed., 1988).

Appropriate chemical modifications of the antisense oligonucleotide inhibitors of the present disclosure can be made to ensure stability of the antisense oligonucleotides, as described below. Changes in the nucleotide sequence and/or in the length of the antisense oligonucleotide can be made to ensure maximum efficiency and thermodynamic stability of the inhibitor. Such sequence and/or length modifications are readily determined by one of ordinary skill in the art.

The antisense oligonucleotides can be DNA or RNA or chimeric mixtures, or derivatives or modified versions thereof, and can be single-stranded or double-stranded. Thus, for example, in the antisense oligonucleotides set forth in herein, when a sequence includes thymidine residues, one or more of the thymidine residues may be replaced by uracil residues and, conversely, when a sequence includes uracil residues, one or more of the uracil residues may be replaced by thymidine residues.

Antisense oligonucleotides comprise sequences complementary to at least a portion of the corresponding target polypeptide. However, 100% sequence complementarity is not required so long as formation of a stable duplex (for single stranded antisense oligonucleotides) or triplex (for double stranded antisense oligonucleotides) can be achieved. The ability to hybridize will depend on both the degree of complementarity and the length of the antisense oligonucleotides. Generally, the longer the antisense oligonucleotide, the more base mismatches with the corresponding nucleic acid target can be tolerated. One skilled in the art can ascertain a tolerable degree of mismatch by use of standard procedures to determine the melting point of the hybridized complex.

Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607).

The skilled artisan will readily appreciate how to design, for an example, an antisense inhibitor of FOXS1, based on the known sequence of the FOXS1 gene. Non-limiting examples of such antisense oligonucleotides include, but are not limited to:

Sense strand:
(SEQ ID NO: 11)
5′ CAUGUGAUGAUGAGGGAAAUU 3′
Antisense strand:
(SEQ ID NO: 12)
3′ UUGUACACUACUACUCCCUUU 5′
Sense strand:
(SEQ ID NO: 13)
5′ GGCUCUAGGACCUGAAGAAUU 3′
Antisense strand:
(SEQ ID NO: 14)
3′ UUCCGAGAUCCUGGACUUCUU 5′
Sense strand:
(SEQ ID NO: 15)
5′ CCAAUAAAGCCAUGUGAUGUU 3′
Antisense strand
(SEQ ID NO: 16)
3′ UUGGUUAUUUCGGUACACUAC 5′

The antisense oligonucleotides can be modified at the base moiety, sugar moiety, or phosphate backbone, or a combination thereof In one embodiment, the antisense oligonucleotide comprises at least one modified sugar moiety, e.g., a sugar moiety such as arabinose, 2-fluoroarabinose, xylulose, and hexose.

In another embodiment, the antisense oligonucleotide comprises at least one modified phosphate backbone such as a phosphorothioate, a phosphorodithioate, a phosphoramidothioate, a phosphoramidate, a phosphordiamidate, a methylphosphonate, an alkyl phosphotriester, and a formacetal or analog thereof. Examples include, without limitation, phosphorothioate antisense oligonucleotides (e.g., an antisense oligonucleotide phosphothioate modified at 3′ and 5′ ends to increase its stability) and chimeras between methylphosphonate and phosphodiester oligonucleotides. These oligonucleotides provide good in vivo activity due to solubility, nuclease resistance, good cellular uptake, ability to activate RNase H, and high sequence selectivity.

Other examples of synthetic antisense oligonucleotides include oligonucleotides that contain phosphorothioates, phosphotriesters, methyl phosphonates, short chain alkyl, or cycloalkyl intersugar linkages or short chain heteroatomic or heterocyclic intersugar linkages. Examples include those with CH2-NH—O—CH2, CH2-N(CH3)-O—CH2, CH2-O—N(CH3)-CH2, CH2-N(CH3)-N(CH3)-CH2 and O—N(CH3)-CH2-CH2 backbones (where phosphodiester is O—PO2-O—CH2). U.S. Pat. No. 5,677,437 describes heteroaromatic oligonucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds.

In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 1991;254:1497). Other synthetic oligonucleotides may contain substituted sugar moieties comprising one of the following at the 2′ position: OH, SH, SCH3, F, OCN, O(CH2)nNH2 or O(CH2)nCH3 where n is from 1 to about 10; C1 to C10 lower alkyl, substituted lower alkyl, alkaryl or aralkyl; Cl; Br; CN; CF3; OCF3; O-; S-, or N-alkyl; O-, S-, or N-alkenyl; SOCH3; SO2CH3; ONO2; NO2; N3; NH2; heterocycloalkyl; heterocycloalkaryl; aminoalkylamino; polyalkylamino; substituted sialyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties.

Oligonucleotides may also have sugar mimetics such as cyclobutyls or other carbocyclics in place of the pentofuranosyl group. Nucleotide units having nucleosides other than adenosine, cytidine, guanosine, thymidine and uridine may be used, such as inosine. In other embodiments, locked nucleic acids (LNA) can be used (reviewed in, e.g., Jepsen and Wengel, Curr. Opin. Drug Discov. Devel. 2004; 7:188-194; Crinelli et al., Curr. Drug Targets 2004; 5:745-752). LNA are nucleic acid analog(s) with a 2′-O, 4′-C methylene bridge. This bridge restricts the flexibility of the ribofuranose ring and locks the structure into a rigid C3-endo conformation, conferring enhanced hybridization performance and exceptional biostability. LNA allows the use of very short oligonucleotides (less than 10 bp) for efficient hybridization in vivo.

In one embodiment, an antisense oligonucleotide can comprise at least one modified base moiety such as a group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-i sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the antisense oligonucleotide can include α-anomeric oligonucleotides. An α-anomeric oligonucleotide forms specific double-stranded hybrids with complementary RNA in which, contrary to the usual β-units, the strands run parallel to each other (Gautier et al., Nucl. Acids Res. 1987; 15:6625-6641).

Oligonucleotides may have morpholino backbone structures (U.S. Pat. No. 5,034,506). Thus, in another embodiment, the antisense oligonucleotide can be a morpholino antisense oligonucleotide (i.e., an oligonucleotide in which the bases are linked to 6-membered morpholine rings, which are connected to other morpholine-linked bases via non-ionic phosphorodiamidate intersubunit linkages). Morpholino oligonucleotides are highly resistant to nucleases and have good targeting predictability, high in-cell efficacy and high sequence specificity (U.S. Pat. No. 5,034,506; Summerton, Biochim. Biophys. Acta 1999; 1489:141-158; Summerton and Weller, Antisense Nucleic Acid Drug Dev. 1997; 7:187-195; Arora et al., J. Pharmacol. Exp. Ther. 2000; 292:921-928; Qin et al., Antisense Nucleic Acid Drug Dev. 2000; 10:11-16; Heasman et al., Dev. Biol. 2000; 222:124-134; Nasevicius and Ekker, Nat. Genet. 2000; 26:216-220).

Antisense oligonucleotides may be chemically synthesized, for example using appropriately protected ribonucleoside phosphoramidites and a conventional DNA/RNA synthesizer. Antisense nucleic acid oligonucleotides can also be produced intracellularly by transcription from an exogenous sequence. For example, a vector can be introduced in vivo such that it is taken up by a cell within which the vector or a portion thereof is transcribed to produce an antisense RNA. Such a vector can remain episomal or become chromosomally integrated, so long as it can be transcribed to produce the desired antisense RNA. Such vectors can be constructed by recombinant DNA technology methods standard in the art. Vectors can be plasmid, viral, or others known in the art, used for replication and expression in mammalian cells. In another embodiment, “naked” antisense nucleic acids can be delivered to adherent cells via “scrape delivery”, whereby the antisense oligonucleotide is added to a culture of adherent cells in a culture vessel, the cells are scraped from the walls of the culture vessel, and the scraped cells are transferred to another plate where they are allowed to re-adhere. Scraping the cells from the culture vessel walls serves to pull adhesion plaques from the cell membrane, generating small holes that allow the antisense oligonucleotides to enter the cytosol.

RNAi

Reversible short inhibition of a target polypeptide (e.g., NCOA4, HERC2, or an ATG8 paralog) of the invention may also be useful. Such inhibition can be achieved by use of siRNAs. RNA interference (RNAi) technology prevents the expression of genes by using small RNA molecules such as small interfering RNAs (siRNAs). This technology in turn takes advantage of the fact that RNAi is a natural biological mechanism for silencing genes in most cells of many living organisms, from plants to insects to mammals (McManus et al., Nature Reviews Genetics, 2002, 3(10) p. 737). RNAi prevents a gene from producing a functional protein by ensuring that the molecule intermediate, the messenger RNA copy of the gene is destroyed siRNAs can be used in a naked form and incorporated in a vector, as described below.

RNA interference (RNAi) is a process of sequence-specific post-transcriptional gene silencing by which double stranded RNA (dsRNA) homologous to a target locus can specifically inactivate gene function in plants, fungi, invertebrates, and vertebrates, including mammals (Hammond et al., Nature Genet. 2001;2:110-119; Sharp, Genes Dev. 1999; 13:139-141). This dsRNA-induced gene silencing is mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200). RNAi-mediated gene silencing is thought to occur via sequence-specific RNA degradation, where sequence specificity is determined by the interaction of a siRNA with its complementary sequence within a target RNA (see, e.g., Tuschl, Chem. Biochem. 2001; 2:239-245).

For mammalian systems, RNAi commonly involves the use of dsRNAs that are greater than 500 bp; however, it can also be activated by introduction of either siRNAs (Elbashir, et al., Nature 2001; 411: 494-498) or short hairpin RNAs (shRNAs) bearing a fold back stem-loop structure (Paddison et al., Genes Dev. 2002; 16: 948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508).

The siRNAs are typically short double stranded nucleic acid duplexes comprising annealed complementary single stranded nucleic acid molecules. Typically, the siRNAs are short dsRNAs comprising annealed complementary single strand RNAs. siRNAs may also comprise an annealed RNA:DNA duplex, wherein the sense strand of the duplex is a DNA molecule and the antisense strand of the duplex is a RNA molecule.

Each single stranded nucleic acid molecule of the siRNA duplex can be of from about 19 nucleotides to about 27 nucleotides in length. In certain embodiments, duplexed siRNAs have a 2 or 3 nucleotide 3′ overhang on each strand of the duplex. In some embodiments, siRNAs have 5′-phosphate and 3′-hydroxyl groups.

Non-limiting examples of RNAi molecules that can be used to inhibit FOXS1 include, e.g.,

Start: 132
(SEQ ID NO: 31)
TCCCTACAGCTACATCGCCCTTATT;
Start: 637
(SEQ ID NO: 32)
CCACCCATGGAGCCCAAAGAGATTT;
and
Start: 1039
(SEQ ID NO: 33)
CGGACGCCAGGAATGTTCTTCTTTG.

RNAi molecules may include one or more modifications, either to the phosphate-sugar backbone or to the nucleoside. For example, the phosphodiester linkages of natural RNA may be modified to include at least one heteroatom other than oxygen, such as nitrogen or sulfur. In this case, for example, the phosphodiester linkage may be replaced by a phosphothioester linkage. Similarly, bases may be modified to block the activity of adenosine deaminase. Where the RNAi molecule is produced synthetically, or by in vitro transcription, a modified ribonucleoside may be introduced during synthesis or transcription. The skilled artisan will understand that many of the modifications described above for antisense oligonucleotides may also be made to RNAi molecules. Such modifications are well known in the art.

siRNAs may be introduced to a target cell as an annealed duplex siRNA, or as single stranded sense and antisense nucleic acid sequences that, once within the target cell, anneal to form the siRNA duplex. Alternatively, the sense and antisense strands of the siRNA may be encoded on an expression construct that is introduced to the target cell. Upon expression within the target cell, the transcribed sense and antisense strands may anneal to reconstitute the siRNA.

shRNAs typically comprise a single stranded “loop” region connecting complementary inverted repeat sequences that anneal to form a double stranded “stem” region. Structural considerations for shRNA design are discussed, for example, in McManus et al., RNA 2002; 8:842-850. In certain embodiments the shRNA may be a portion of a larger RNA molecule, e.g., as part of a larger RNA that also contains U6 RNA sequences (Paul et al., supra).

In some embodiments, the loop of the shRNA is from about 1 to about 9 nucleotides in length. In some embodiments the double stranded stem of the shRNA is from about 19 to about 33 base pairs in length. In some embodiments, the 3′ end of the shRNA stem has a 3′ overhang. In some embodiments, the 3′ overhang of the shRNA stem is from 1 to about 4 nucleotides in length. In some embodiments, shRNAs have 5′-phosphate and 3′-hydroxyl groups.

Non-limiting, exemplary shRNA sequences targeted to FOXS1, include, e.g.:

FOXS1 shRNA_a
Top:
(SEQ ID NO: 34)
GCCAGGAATGTTCTTCTTTGTTCAAGAGACAAAGAAGAACATTCCT
GGCTTTTTTGT;
Bottom:
(SEQ ID NO: 35)
CTAGACAAAAAAGCCAGGAATGTTCTTCTTTGTCTCTTGAACAAAG
AAGAACATTCCTGGC;
FOXS1 shRNA_b
Top:
((SEQ ID NO: 36)
GCCAATAAAGCCATGTGATTTCAAGAGAATCACATGGCTTTATTGG
CTTTTTTGT;
Bottom:
(SEQ ID NO: 37)
CTAGACAAAAAAGCCAATAAAGCCATGTGATTCTCTTGAAATCACA
TGGCTTTATTGGC;
and
FOXS1 shRNA_C
Top:
((SEQ ID NO: 38)
GCATCTACCGCTACATCATTTCAAGAGAATGATGTAGCGGTAGATG
CTTTTTTGT;
Bottom:
((SEQ ID NO: 39)
CTAGACAAAAAAGCATCTACCGCTACATCATTCTCTTGAAATGATG
TAGCGGTAGATGC.

Other examples of shRNA sequences include, e.g., those exemplified in Example 5, below:

FoxS1 shRNAa
(SEQ ID NO: 50)
(GCCAGGAATGTTCTTCTTTG);
FoxS1 shRNAb
(SEQ ID NO: 51)
(GCCAATAAAGCCATGTGAT);
and
FoxS1 shRNAc
(SEQ ID NO: 52)
(GCATCTACCGCTACATCAT).

The skilled artisan will readily appreciate how to design and test other candidate shRNA molecules targeted to FOXS 1 and/or other targets encompassed by the present methods.

Although RNAi molecules can contain nucleotide sequences that are fully complementary to a portion of the target nucleic acid, 100% sequence complementarity between the RNAi probe and the target nucleic acid is not required.

Similar to the above-described antisense oligonucleotides, RNAi molecules can be synthesized by standard methods known in the art, e.g., by use of an automated synthesizer. RNAs produced by such methodologies tend to be highly pure and to anneal efficiently to form siRNA duplexes or shRNA hairpin stem-loop structures. Following chemical synthesis, single stranded RNA molecules are deprotected, annealed to form siRNAs or shRNAs, and purified (e.g., by gel electrophoresis or HPLC). Alternatively, standard procedures may be used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donzé and Picard, Nucleic Acids Res. 2002; 30:e46; and Yu et al., Proc. Natl. Acad. Sci. USA 2002; 99:6047-6052). Similarly, an efficient in vitro protocol for preparation of shRNAs using T7 RNA polymerase has been described (Yu et al., supra). The sense and antisense transcripts may be synthesized in two independent reactions and annealed later, or may be synthesized simultaneously in a single reaction.

RNAi molecules may be formed within a cell by transcription of RNA from an expression construct introduced into the cell. For example, both a protocol and an expression construct for in vivo expression of siRNAs are described in Yu et al., supra. The delivery of siRNA to tumors can potentially be achieved via any of several gene delivery “vehicles” that are currently available. These include viral vectors, such as adenovirus, lentivirus, herpes simplex virus, vaccinia virus, and retrovirus, as well as chemical-mediated gene delivery systems (for example, liposomes), or mechanical DNA delivery systems (DNA guns). The oligonucleotides to be expressed for such siRNA-mediated inhibition of gene expression would be between 18 and 28 nucleotides in length. Protocols and expression constructs for in vivo expression of shRNAs have been described (Brummelkamp et al., Science 2002; 296:550-553; Sui et al., supra; Yu et al., supra; McManus et al., supra; Paul et al., supra).

The expression constructs for in vivo production of RNAi molecules comprise RNAi encoding sequences operably linked to elements necessary for the proper transcription of the RNAi encoding sequence(s), including promoter elements and transcription termination signals. Exemplary promoters for use in such expression constructs include the polymerase-III HI-RNA promoter (see, e.g., Brummelkamp et al., supra) and the U6 polymerase-III promoter (see, e.g., Sui et al., supra; Paul, et al. supra; and Yu et al., supra). The RNAi expression constructs can further comprise vector sequences that facilitate the cloning of the expression constructs. Standard vectors are known in the art (e.g., pSilencer 2.0-U6 vector, Ambion Inc., Austin, Tex.).

Ribozyme Inhibition

The level of expression of a target polypeptide of the invention can also be inhibited by ribozymes designed based on the nucleotide sequence thereof.

Ribozymes are enzymatic RNA molecules capable of catalyzing the sequence-specific cleavage of RNA (for a review, see Rossi, Current Biology 1994;4:469-471). The mechanism of ribozyme action involves sequence-specific hybridization of the ribozyme molecule to complementary target RNA, followed by an endonucleolytic cleavage event. The composition of ribozyme molecules must include: (i) one or more sequences complementary to the target RNA; and (ii) a catalytic sequence responsible for RNA cleavage (see, e.g., U.S. Pat. No. 5,093,246).

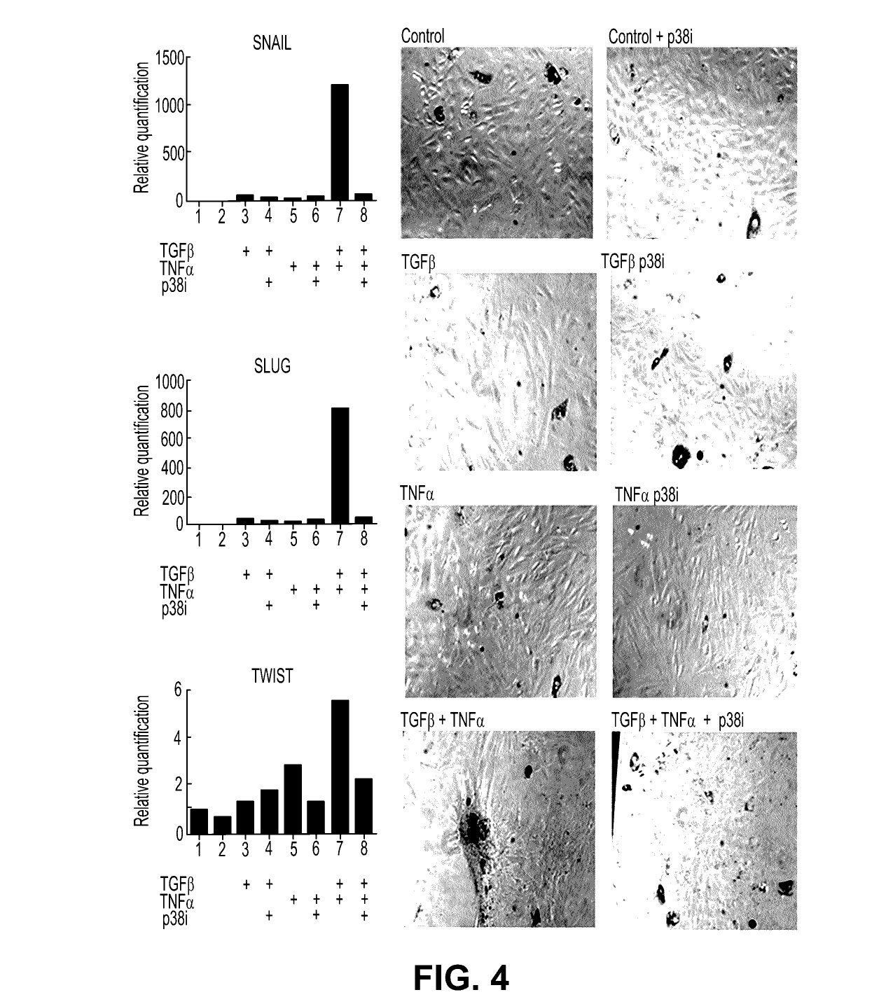

The use of hammerhead ribozymes is contemplated. Hammerhead ribozymes cleave RNAs at locations dictated by flanking regions that form complementary base pairs with the target RNA. The sole requirement is that the target RNA has the following sequence of two bases: 5′-UG-3′. The construction of hammerhead ribozymes is known in the art, and described more fully in Myers, Molecular Biology and Biotechnology: A Comprehensive Desk Reference, VCH Publishers, New York, 1995 (see especially FIG. 4, page 833) and in Haseloff and Gerlach, Nature 1988; 334:585-591.

As in the case of antisense oligonucleotides, ribozymes can be composed of modified oligonucleotides (e.g., for improved stability, targeting, etc.). These can be delivered to cells which express the target polypeptide in vivo. An exemplary method of delivery involves using a DNA construct “encoding” the ribozyme under the control of a strong constitutive pol III or pol II promoter, so that transfected cells will produce sufficient quantities of the ribozyme to catalyze cleavage of the target mRNA encoding the target polypeptide. However, because ribozymes, unlike antisense molecules, are catalytic, a lower intracellular concentration may be required to achieve an adequate level of efficacy.

Ribozymes can be prepared by any method known in the art for the synthesis of DNA and RNA molecules, as discussed above. Ribozyme technology is described further in Intracellular Ribozyme Applications: Principals and Protocols, Rossi and Couture eds., Horizon Scientific Press, 1999.

Triple Helix Forming Oligonucleotides (TFOs)

Nucleic acid molecules useful to inhibit expression level of a target polypeptide of the invention via triple helix formation are typically composed of deoxynucleotides. The base composition of these oligonucleotides is typically designed to promote triple helix formation via Hoogsteen base pairing rules, which generally require sizeable stretches of either purines or pyrimidines to be present on one strand of a duplex. Nucleotide sequences may be pyrimidine-based, resulting in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., those containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, sequences can be targeted for triple helix formation by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Similarly to RNAi molecules, antisense oligonucleotides, and ribozymes, described above, triple helix molecules can be prepared by any method known in the art. These include techniques for chemically synthesizing oligodeoxyribonucleotides and oligoribonucleotides such as, e.g., solid phase phosphoramidite chemical synthesis. Alternatively, RNA molecules can be generated by in vitro or in vivo transcription of DNA sequences “encoding” the particular RNA molecule. Such DNA sequences can be incorporated into a wide variety of vectors that incorporate suitable RNA polymerase promoters such as the T7 or SP6 polymerase promoters. See, Nielsen, P. E. “Triple Helix: Designing a New Molecule of Life”, Scientific American, December, 2008; Egholm, M., et al. “PNA Hybridizes to Complementary Oligonucleotides Obeying the Watson-Crick Hydrogen Bonding Rules.” (1993) Nature, 365, 566-568; Nielsen, P.E. ‘PNA Technology’. Mol Biotechnol. 2004; 26:233-48.

Aptamers

Aptamers are oligonucleic acid or peptide molecules that bind to a specific target molecule. Aptamers can be used to inhibit gene expression and to interfere with protein interactions and activity. Nucleic acid aptamers are nucleic acid species that have been engineered through repeated rounds of in vitro selection (e.g., by SELEX (systematic evolution of ligands by exponential enrichment)) to bind to various molecular targets such as small molecules, proteins, nucleic acids, and even cells, tissues and organisms. Peptide aptamers consist of a variable peptide loop attached at both ends to a protamersein scaffold. Aptamers are useful in biotechnological and therapeutic applications as they offer molecular recognition properties that rival that of antibodies. Aptamers can be designed and used to inhibit a polypeptide disclosed herein, e.g., p38 and/or another p38 pathway signaling molecule, and /orFOXS 1 and/or another FOXS 1 signaling pathway molecule.

Aptamers can be engineered completely in a test tube, are readily produced by chemical synthesis, possess desirable storage properties, and elicit little or no immunogenicity in therapeutic application. Aptamers can be produced using the methodology disclosed in a U.S. Pat. No. 5,270,163 and WO 91/19813. See also Kanwar et al. Drug Discov Today. 2014 Mar. 2. pii: S1359-6446(14)00062-2. doi: 10.1016/j.drudis.2014.02.009 (E-Pub ahead of print); Cunningham et al. Ophthalmology. 2005 October; 112(10): 1747-57; Santosh and Yadava, Biomed Res Int. 2014; 2014:540451; Xing et al. Curr Opin Chem Eng. 2014 May 1; 4:79-87.

### Antibodies

Blocking antibodies can be used to inhibit a polypeptide disclosed herein, e.g., p38 and/or another p38 pathway signaling molecule, and/or FOXS1 or another FOXS1 signaling pathway molecule. By way of example, commercially available antibodies to p38 and FOXS1 are available, e.g., from Abcam, Lifespan, SantaCruz Biotech. Moreover, methods for designing and screening an antibody for use in the methods disclosed herein are routine in the art.

Antibodies, or their equivalents and derivatives, e.g., intrabodies, or other antagonists of the polypeptide, may be used in accordance with the present methods. Methods for engineering intrabodies (intracellular single chain antibodies) are well known. Intrabodies are specifically targeted to a particular compartment within the cell, providing control over where the inhibitory activity of the treatment is focused. This technology has been successfully applied in the art (for review, see Richardson and Marasco, 1995, TIBTECH vol. 13; Lo et al. (2009) Handb Exp Pharmacol. 181:343-73; Maraasco, W. A. (1997) Gene Therapy 4:11-15; see also, U.S. Pat. Appln. Pub. No. 2001/0024831 by Der Maur et al. and U.S. Pat. No. 6,004,940 by Marasco et al.).

Administration of a suitable dose of the antibody or the antagonist (e.g., aptamer) may serve to block the level (expression or activity) of the polypeptide in order to treat or prevent a disease or condition disclosed herein (e.g., p38 or FOXS1).

In addition to using antibodies and aptamers to inhibit the level and/or activity of a target polypeptide, it may also be possible to use other forms of inhibitors. For example, it may be possible to identify antagonists that functionally inhibit the target polypeptide (e.g., p38, FOXS1). In addition, it may also be possible to interfere with the interaction of the polypeptide with its substrate. Other suitable inhibitors will be apparent to the skilled person.

The antibody (or other inhibitors and antagonists) can be administered by a number of methods. For example, for the administration of intrabodies, one method is set forth by Marasco and Haseltine in PCT WO 94/02610. This method discloses the intracellular delivery of a gene encoding the intrabody. In one embodiment, a gene encoding a single chain antibody is used. In another embodiment, the antibody would contain a nuclear localization sequence. By this method, one can intracellularly express an antibody, which can block activity of the target polypeptide in desired cells (e.g., RPE cells or breast epithelial cells).

Peptide Inhibitors

Also contemplated for use herein are peptide inhibitors of the FOXS1 signaling pathway. By way of example, a peptide inhibitor can be used to interfere with FOXS1 signaling. In a specific embodiment, a peptide inhibitor can interfere with p38 signaling.

Small Molecules

Chemical agents, referred to in the art as “small molecule” compounds are typically organic, non-peptide molecules, having a molecular weight less than 10,000 Da, preferably less than 5,000 Da, more preferably less than 1,000 Da, and most preferably less than 500 Da. This class of modulators includes chemically synthesized molecules, for instance, compounds from combinatorial chemical libraries. Synthetic compounds may be rationally designed or identified utilizing the screening methods described below. Methods for generating and obtaining small molecules are well known in the art (Schreiber, Science 2000; 151:1964-1969; Radmann et al., Science 2000; 151:1947-1948). Non-limiting examples of small molecule inhibitors encompassed by the methods disclosed herein include the p38 inhibitor SB202190. As disclosed above, other exemplary small molecule inhibitors include, e.g., AMG548 (p38 MAPK inhibitor), AS1940477 (p38 MAP kinase inhibitor), CBS3830 (p38 MAPK inhibitor), Dilmapimod|SB-6813123 (p38 MAP kinase inhibitor), Doramapimod|BIRB-796 (p38 MAPK inhibitor), FR-167653 (p38 MAPK inhibitor), JLU1124 (p38 MAPK inhibitor), LASSBio-998 (p38 MAPK inhibitor), Losmapimod (GW856553) (p38 MAP kinase inhibitor), LY2228820 (p38 MAP kinase inhibitor), LY3007113 (p38 MAP kinase inhibitor), ML3403 (p38 MAP kinase inhibitor), Pamapimod (p38 MAP kinase inhibitor), PD-98059|PD098059 (p38 MAP kinase inhibitor), PD-169316 (p38 MAP kinase inhibitor), PH-797804 (p38 MAP kinase inhibitor), R-130823 (p38 MAP kinase inhibitor), RO3201195 (p38 MAP kinase inhibitor), RPR-200765A (p38 MAP kinase inhibitor), RPR-203494(p38 MAP kinase inhibitor), RWJ-67657 (p38 MAP kinase inhibitor), SB-203580 (p38 MAP kinase inhibitor), SB-239063 (p38 MAP kinase inhibitor), SB-242235 (p38 MAPK inhibitor), SCID-323 (p38 MAP kinase inhibitor), SD-282 (p38 MAPK inhibitor), Semapimod|CNI-1493 (p38 MAPK inhibitor), Soblidotin|TZT-1027 (p38 MAPK inhibitor), TAK-715 (p38 MAPK inhibitor), Talmapimod|SCID-469 (p38 MAPK inhibitor), UO126 (p38 MAPK inhibitor), UR-13756 (p38 MAPK inhibitor), VX-702 (p38 MAPK inhibitor), VX-745 (p38 MAPK inhibitor), and nicotinamide.

Other suitable small molecules and methods for screening for suitable small molecule inhibitors are known in the art, and described below (see “screening methods”).

In certain embodiments, the above described inhibitors and agonists can be directly targeted to a specific cell type (e.g., RPE cell) or to a site of EMT (e.g., the eye, the skin, breast epithelium). The skilled artisan will appreciate that methods for specific cell targeting are well known in the art. By way of non-limiting example, antibodies targeted to the desired cell type may be conjugated to an inhibitor or agonist described herein, in order to target the inhibitor or agonist to, for example and without limitation, an RPE cell and/or an RPE cells undergoing EMT. Further, the site of administration (e.g., direct injection into the RPE layer or topical administration to the retina or other epithelia can further increase the specificity of cell targeting.

### Screening Methods

In certain embodiments, the present disclosure provides methods for screening for compounds that inhibit EMT in epithelial cells.

In some embodiments, the method can include providing a monolayer of epithelial cells; culturing the monolayer in conditions that induce EMT (e.g., in the presence of TNFα and TGFβ); contacting the monolayer of cells with a test compound (either before, at the same time as, or after inducing EMT); measuring the expression level of at least one member of the FOXS1 signaling pathway; and identifying the test compound as a candidate inhibitor of EMT if the expression level of the at least one member of the FOXS1 signaling pathway is decreased relative to a control or reference level. In some embodiments, the method of screening includes providing a monolayer of epithelial cells; culturing the monolayer in conditions that induce EMT (e.g., culturing the cells in the presence of TNFα and TGFβ); contacting the monolayer of cells with a test compound (either before, at the same time as, or after inducing EMT); measuring the expression level of at least one EMT-associated marker selected from FOXS1, SLUG, SNAIL, and TWIST; and identifying the test compound as a candidate inhibitor of EMT if the expression level of the at least one marker is decreased relative to a control or reference level. In some embodiments, the mRNA expression level of the marker is determined. In other embodiments, the protein expression level of the marker is determined.

In some embodiments, the epithelial cells are RPE cells, and the method comprises or further comprises determining the expression levels of one or more markers of RPE cells. Exemplary markers of RPE cells include, e.g., OTX2, Bestrophin, RPE65, Mitf, and Cralbp. See, U.S. Pat. No. 8,481,313 by Temple et al. In some embodiments, a test compound is determined to be a suitable candidate inhibitor of FOXS1 signaling pathway, i.e., a suitable candidate inhibitor of EMT, if the level of the one or more markers of RPE cells is increased compared to control levels (e.g., cells undergoing EMT in the absence of the test compound). For example, a test compound may be determined to be effective more inhibiting EMT if the expression level of one or both of OTX2 and Bestrophin in the subject is increased.

The skilled artisan will readily appreciate that dosage escalation studies can also be performed for a test compound to identify effective dosages.

Methods for providing the monolayer of epithelial cells are known in the art. Described herein is a method of producing human RPE monolayers from human retinal pigment epithelial stem cells (RPESCs), as described in Example 1, below. However, the skilled artisan will readily appreciate that other types and/or sources of epithelial cells or progenitor cells (i.e., multipotent cells that can be induced to differentiate into RPE cells, e.g., embryonic stem cells, induced pluripotent stem cells, parthogenic stem cells, and tissue specific epithelial stem cells can be used to produce suitable epithelial monolayers in culture. For example, also described herein is the production of epithelial monolayers from the breast epithelial cell line, hTERT HMEnt (HME) (see Example 8). Non-limiting examples of epithelial cell lines that can be used to screen candidate inhibitors of EMT include but are not limited to epithelial cell lines produced from mammary, alveolar, bronchial, colonic, esophageal, renal, liver, ovarian, pancreatic, prostatic, intestinal, splenic, thyroid, and the like. Such cell lines are commercially available.

It is presently discovered that the combination of TGFβ and TNFα (“TnT”) induces upregulation of EMT-related transcripts in RPE cells, including the transcripts for SNAIL, SLUG and TWIST. As discussed above, for RPE cells, specifically, the levels of RPE specific markers can be determined, e.g., OTX2, Bestrophin, Mitf, Cralbp. The human markers have the GenBank® Accession Nos. shown in Table 2, below.

TABLE 2
EMT-related and RPE Markers

| | SEQ | GenBank ® | SEQ | GenBank ® |
|---|---|---|---|---|
| | ID | Accession No. | ID | Accession No. |
| EMT-related Transcript | NO. | (nucleic acid) | NO. | (protein) |
| SNAIL - snail family | 17 | NM_005985 | 18 | NP_005976 |
| zinc finger 1 | | | | |
| (SNAI1) | | | | |
| SLUG - snail family | 19 | NM_003068 | 20 | NP_003059 |
| zinc finger 2 | | | | |
| (SNAI2) | | | | |
| TWIST - twist basic | 21 | NM_003068 | 22 | NP_003059 |
| helix-loop-helix | | | | |
| transcription | | | | |
| factor 1 | | | | |
| OTX2 - orthodenticle | 23 | NM_021728 | 24 | NP_068374 |
| homeobox 2 | | | | |
| Bestrophin | 25 | AF057170 | 26 | AAC64344 |
| Mitf - microphthalmia- | 27 | NM_198159^{a} | 28 | NP_937802^{b} |
| associated transcription | | | | |
| factor | | | | |
| Cralbp - retinaldehyde- | 29 | NM_000326 | 30 | NP_000317 |
| binding protein | | | | |

^{a}see also Accession No. NM_198178;
^{b}see also Accession No. NP_937821

The skilled artisan will readily appreciate how to measure the mRNA and/or protein levels of EMT-associated markers (e.g., FOXS1, SNAIL, SLUG, TWIST) and/or of RPE-specific markers (e.g., OTX2, Bestrophin, Mitf, Cralbp), since the sequences and proteins structures are known in the art (see, e.g., Martinez-Morales et al., Bioessays (2004) 26:766-777; Ohno-Matsui et al., Mol. Vis. (2005) 11:1-10 for a description of RPE markers). However, non-limiting examples of suitable methods for determining expression levels of EMT-associated markers and RPE markers are described in the following section.

### Methods for Determining Expression Levels

In certain embodiments, it is desirable to determine (e.g., assay, measure, approximate) the level (e.g., expression and/or activity), of an EMT-associated marker. The expression level of such markers may be determined according to any suitable method known in the art. A non-limiting example of such a method includes real-time PCR (RT-PCR), e.g., quantitative RT-PCR (QPCR), which measures the expression level of the mRNA encoding the polypeptide. Real-time PCR evaluates the level of PCR product accumulation during amplification. RNA (or total genomic DNA for detection of germline mutations) is isolated from a sample. RT-PCR can be performed, for example, using a Perkin Elmer/Applied Biosystems (Foster City, Calif.) 7700 Prism instrument. Matching primers and fluorescent probes can be designed for genes of interest using, based on the genes' nucleic acid sequences (e.g., as described above), for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.).

To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes. Methods of QPCR using TaqMan probes are well known in the art. Detailed protocols for QPCR are provided, for example, for RNA in: Gibson et al., 1996, Genome Res., 10:995-1001; and for DNA in: Heid et al., 1996, Genome Res., 10:986-994; and in Innis et al. (1990) Academic Press, Inc. N.Y.

Expression of mRNA, as well as expression of peptides and other biological factors can also be determined using microarray, methods for which are well known in the art [see, e.g., Watson et al. Curr Opin Biotechnol (1998) 9: 609-14; “DNA microarray technology: Devices, Systems, and Applications” Annual Review of Biomedical Engineering; Vol. 4: 129-153 (2002); Chehab et al. (1989) “Detection of specific DNA sequences by fluorescence amplification: a color complementation assay” Proc. Natl. Acad. Sci. USA, 86: 9178-9182; Lockhart et al. (1996) “Expression monitoring by hybridization to high-density oligonucleotide arrays” Nature Biotechnology, 14: 1675-1680; and M. Schena et al. (1996) “Parallel human genome analysis: Microarray-based expression monitoring of 1000 genes” Proc. Natl. Acad. Sci. USA, 93:10614-10619; Peptide Microarrays Methods and Protocols; Methods in Molecular Biology; Volume 570, 2009, Humana Press; and Small Molecule Microarrays Methods and Protocols; Series: Methods in Molecular Biology, Vol. 669, Uttamchandani, Mahesh; Yao, Shao Q. (Eds.) 2010, 2010, Humana Press]. For example, mRNA expression profiling can be performed to identify differentially expressed genes, wherein the raw intensities determined by microarray are loge-transformed and quantile normalized and gene set enrichment analysis (GSEA) is performed according, e.g., to Subramanian et al. (2005) Proc Nall Acad Sci USA 102:15545-15550).

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc. In another embodiment, DNA sequencing may be used to determine the presence of ER in a genome. Methods for DNA sequencing are known to those of skill in the art.

Other methods for detecting gene expression (e.g., mRNA levels) include Serial Analysis of Gene Expression applied to high-throughput sequencing (SAGEseq), as described in Wu Z J et al. Genome Res. 2010 December; 20(12):1730-9. 2.

Methods for detecting the expression levels of polypeptides are also known in the art. Non-limiting examples of suitable methods for detecting expression levels of gene products (i.e., polypeptides) described herein include, e.g., flow cytometry, immunoprecipitation, Western blot (see, e.g., Battle T E, Arbiser J, & Frank D A (2005) Blood 106(2):690-697), ELISA (enzyme-linked immunosorbent assay), multiplex assay, and/or immunohistochemistry.

### Compositions and Formulations

In certain embodiments, provided herein is a method for treating a disease or disorder associated with EMT. The method can include administering to a subject a composition comprising an inhibitor of the FOXS1 signaling pathway.

While it is possible to use an agent or composition disclosed herein for therapy as is, it may be preferable to administer an inhibitor or agonist as a pharmaceutical formulation, e.g., in admixture with a suitable pharmaceutical excipient, diluent, or carrier selected with regard to the intended route of administration and standard pharmaceutical practice. Pharmaceutical formulations comprise at least one active compound, or a pharmaceutically acceptable derivative thereof, in association with a pharmaceutically acceptable excipient, diluent, and/or carrier. The excipient, diluent and/or carrier must be “pharmaceutically acceptable.”

In certain embodiments, provided herein is a pharmaceutical formulation that contains (a) an effective amount for inhibiting the FOXS1 signaling pathway of an inhibitor such as an antisense oligonucleotide, a small molecule, a peptide, or a ribozyme, and (b) a pharmaceutical carrier. In certain embodiments, the inhibitor reduces FOXS1 expression level and/or activity when administered to a subject suffering from EMT. Thus, in some embodiments, the pharmaceutical formulation is for use in the treatment of a disease or disorder associated with EMT, e.g., a disease or disorder such as ERM formation, macular pucker, or PVR. The disease or disorder can also be abnormal breast epithelial cell growth, e.g., breast cancer.

Thus, in some embodiments, a composition or pharmaceutical formulation disclosed herein contains an inhibitor of FOXS1 (e.g., a FOXS1 inhibitor disclosed herein) or an inhibitor of another mediator of the FOXS1 signaling pathway. In other embodiments, a composition or pharmaceutical formulation disclosed herein contains an inhibitor of the p38 signaling pathway, e.g., a p38 antagonist. In a specific embodiment, the inhibitor is the small molecule p38 inhibitor SB202190. In another embodiment the inhibitor is a small molecule inhibitor such as, e.g., AMG548 (p38 MAPK inhibitor), AS1940477 (p38 MAP kinase inhibitor), CBS3830 (p38 MAPK inhibitor), Dilmapimod|SB-6813123 (p38 MAP kinase inhibitor), Doramapimod|BIRB-796 (p38 MAPK inhibitor), FR-167653 (p38 MAPK inhibitor), JLU1124 (p38 MAPK inhibitor), LASSBio-998 (p38 MAPK inhibitor), Losmapimod (GW856553) (p38 MAP kinase inhibitor), LY2228820 (p38 MAP kinase inhibitor), LY3007113 (p38 MAP kinase inhibitor), ML3403 (p38 MAP kinase inhibitor), Pamapimod (p38 MAP kinase inhibitor), PD-98059|PD098059 (p38 MAP kinase inhibitor), PD-169316 (p38 MAP kinase inhibitor), PH-797804 (p38 MAP kinase inhibitor), R-130823 (p38 MAP kinase inhibitor), RO3201195 (p38 MAP kinase inhibitor), RPR-200765A (p38 MAP kinase inhibitor), RPR-203494(p38 MAP kinase inhibitor), RWJ-67657 (p38 MAP kinase inhibitor), SB-203580 (p38 MAP kinase inhibitor), SB-239063 (p38 MAP kinase inhibitor), SB-242235 (p38 MAPK inhibitor), SCIO-323 (p38 MAP kinase inhibitor), SD-282 (p38 MAPK inhibitor), Semapimod|CNI-1493 (p38 MAPK inhibitor), Soblidotin|TZT-1027 (p38 MAPK inhibitor), TAK-715 (p38 MAPK inhibitor), Talmapimod|SCIO-469 (p38 MAPK inhibitor), UO126 (p38 MAPK inhibitor), UR-13756 (p38 MAPK inhibitor), VX-702 (p38 MAPK inhibitor), VX-745 (p38 MAPK inhibitor), or nicotinamide.

The compositions disclosed herein can also be formulated as sustained release compositions. By way of example, a composition can be formulated with a biodegradable material such as poly(lactic-co-glycolic acid) or “PLGA” for sustained release. Exemplary suitable sustained release compositions are described, e.g., in U.S. Patent Publication No. 2010/0021422 by Temple et al. Another exemplary sustained release composition, ethylene-vinyl acetate copolymer pellets, which can be loaded with a desired agent (e.g. an inhibitor of FOXS1 signaling pathway, or the p38 signaling pathway, described herein), is described in Ozaki et al. Exp Eye Res. 1997 April; 64(4):505-17. Also encompassed by the methods disclosed herein are delivery methods including intravitreal implants, nanoparticulate carriers, viral vectors and sonotherapy. See Thakur et al. Expert Opin Drug Deliv. 2014 Jun. 14:1-16; Lambiase et al. Drugs Today (Barc). 2014 March; 50(3):239-49; and Boddu et al. Recent Pat Drug Deliv Formul. 2014 April; 8(1):27-36.

### Administration, Dosage and Treatment

Compositions and formulations comprising an inhibitor/antagonist or agonist (i.e., an “agent”) disclosed herein (e.g., an inhibitor/antagonist of the FOXS1 and/or p38 signaling pathway), can be administered by any suitable route of administration known in the art. For example, and without limitation, suitable routes of administration include, e.g., topical (e.g., application to the skin (e.g., topical cream) or eye (e.g., application to retina or cornea, e.g., eye drops), parenteral, and mucosal. The term “parenteral” includes injection (for example, intravenous, intraperitoneal, intramuscular, intraluminal, intratracheal, subcutaneous, intravitreal (e.g., injection into RPE layer, injection into retina, or other part of the eye. Other exemplary routes of administration include, e.g., an implantable delivery device (e.g., subcutaneously implanted devices or implanted intravitreal devices, e.g., as discussed above).

It will be appreciated that the amount of an inhibitor required for use in treatment will vary with the route of administration, the nature of the condition for which treatment is required, and the age, body weight and condition of the patient, and will be ultimately at the discretion of the attendant physician or veterinarian. Compositions will typically contain an effective amount of the active agent(s), alone or in combination. Preliminary doses can be determined according to animal tests, and the scaling of dosages for human administration can be performed according to art-accepted practices.

Therapeutically effective dosages can be determined stepwise by combinations of approaches such as (i) characterization of effective doses of the composition or compound in in vitro cell culture assays using level of inhibition of EMT in the RPE model described herein as a readout followed by (ii) characterization in animal studies (e.g. an animal model of PVR and EMT), followed by (iii) characterization in human trials using improvement in one or more symptoms of a disease associated with EMT (e.g., ERM formation, PVR, macular pucker) as a readout.

Length of treatment, i.e., number of days, will be readily determined by a physician treating the subject; however the number of days of treatment may range from 1 day to about 20 days. Administration of a composition or formulation can be once a day, twice a day, or more often. Frequency may be decreased during a treatment maintenance phase of the disease or disorder, e.g., once every second or third day instead of every day or twice a day. The dose and the administration frequency will depend on the clinical signs, which confirm maintenance of the remission phase, with the reduction or absence of at least one or more, preferably more than one, clinical signs of the acute phase known to the person skilled in the art. More generally, dose and frequency will depend in part on recession of pathological signs and clinical and subclinical symptoms of a disease condition or disorder contemplated for treatment with the present compounds.

In one embodiment, a subject in need of treatment is administered a single injection to the posterior of the eye of a composition disclosed herein (e.g. a FOXS1 signaling pathway inhibitor, e.g., a FOXS1 antagonists, or a p38 signaling pathway inhibitor, e.g., a p38 antagonist). In other embodiments, multiple injections of the composition are administered, e.g., daily, every other day, every third day, every fourth day, every fifth day, every sixth day, weekly, twice per week, thrice per week, etc., over a period of 1 day, 2 days, 3 days, 4 days, 5 days, 6 days, 1 week, 2 weeks, 3 weeks, 4 weeks, 5 weeks, 6 weeks, 7 weeks, 8 weeks, or longer, e.g., over a period of 3 months, 4 months, 5 months, 6 months, 7 months, 8 months, 9 months, 10 months 11 months, 1 year, 2 years, or longer.

As provided by the present methods, and discussed below, the efficacy of treatment can be monitored during the course of treatment to determine whether the treatment has been successful, or whether additional (or modified) treatment is necessary.

Typically, when an antagonist/inhibitor of the present disclosure is administered as a therapy (e.g., for inhibiting EMT and/or for treating a disease or disorder associated with EMT formation), the therapy is deemed effective if the level or activity of the target molecule (e.g., FOXS1, p38, or other mediator of a FOXS1 or p38 signaling pathway (or other signaling pathway that regulates FOXS1 expression and/or activity)) is decreased by at least 1.5-fold, at least 2-fold, at least 3-fold, at least 4-fold, at least 5-fold, at least 10-fold, at least 50-fold, at least 100-fold, or more, relative to the level of the target gene or polypeptide at the beginning of or before commencement of the therapy. The target can be the target gene transcript and/or the encoded polypeptide.

Treatment of ERM, PVR and macular pucker can be monitored by direct observation of the ERM using, for example and without limitation, an ophthalmoscope, fundus camera, B-scan ultrasound and/or optical coherence tomography (OCT). Indirect measures of ERM, PVR and macular pucker include visual acuity, visual field sensitivity, retinal attachment status, metamorphopsia (e.g., on an Amsler grid), dark adaptometry and other measures of visual function.

In other embodiments, provided herein is a method of inhibiting EMT in epithelial cells in a subject suffering from a disease or disorder associated with EMT, wherein the method includes administering the subject an inhibitor of one or both of TGFβ and TNFα. Preferably one or more inhibitors of both TGFβ and TNFα are administered to the subject. Inhibitors of both TGFβ and TNFα are known in the art. For example, an inhibitory antibody targeted to each of TGFβ and TNFα can be administered to the subject. The subject may then be monitored to determine if EMT is inhibited.

Kits

In certain embodiments, kits are provided for diagnosing EMT. In other embodiments, kits are provided for treating EMT or a disease or disorder associated with EMT (e.g., ERM formation, macular pucker, PVR, breast cancer).

The above kits can contain means (e.g., reagents, dishes, solid substrates (e.g., microarray slides, ELISA plates, multiplex beads), solutions, media, buffers, etc.) for determining the level of expression or activity of one or more of the markers (genes or proteins) described herein. For example, for detecting/diagnosing EMT in a subject, the kit can contain reagents for determining the expression levels of FOXS1 and/or one or more of the EMT-associated markers SLUG, SNAIL, and TWIST, in a sample (e.g. biopsy) obtained from the subject.

In some embodiments, the kits contain PCR primers for detecting the above-described markers. Methods for designing primers are known in the art, and are routine when the nucleic acid sequences for the target markers are known, as they are here. The kits can also contain, alternatively or in addition, reagents for detecting protein expression of the markers, such as FOXS1 and/or one or more of SLUG, SNAIL and TWIST, e.g., for determining protein expression by ELISA, multiplex assay, Western blot, or other suitable method known in the art.

In other embodiments, kits for treating EMT can include an inhibitor of the FOXS1 signaling pathway. In a preferred embodiment, the kit provides an inhibitor of FOXS1. For example, as disclosed herein, a FOXS1 inhibitor can be an antisense oligonucleotide, a small molecule, a peptide, or a ribozyme, as disclosed herein.

In other embodiments, the kits comprise inhibitors (e.g., inhibitory antibodies or small molecules) of TGFβ and TNFα.

In some embodiments, the kit includes reagents for both diagnosing EMT, as disclosed above, and for treating EMT, as disclosed above.

Such kits can further comprise instructions for use, e.g., guidelines for diagnosing and/or treating EMT in a subject, based on the level of expression and/or activity of the one or more markers (e.g., FOXS1, SNAIL, SLUG, and/or TWIST) detected using the kit and/or based on the subject's diagnosis (diagnosed with EMT, and/or a disease or disorder associated with EMT, such as, e.g., ERM formation, PVR, macular pucker, breast cancer) as determined by the subject's physician.

The kits, regardless of type, will generally comprise one or more containers into which the biological agents (e.g., detection reagents, inhibitors) are placed and, preferably, suitably aliquotted. The components of the kits may be packaged either in aqueous media or in lyophilized form. The kits can also comprise one or more pharmaceutically acceptable excipients, diluents, and/or carriers.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### EXAMPLES

### Example 1

### RPE Cells Culture

This Examples describes the design of an in vitro model of EMT based on RPE cells produced from RPESCs.

The pathophysiology of ERM formation after displacement of RPE cells from their niche on Bruchs membrane involves epithelial to mesenchymal transition (EMT). After RPE cells undergo EMT, they proliferate to form myocontractile fibrous ERM. This process was recapitulated in cell culture to develop a system in which isolated RPE cells undergo EMT to form myocontractile fibrous membranes in vitro that closely resemble ERMs in patients.

This in vitro EMT model is based on RPE cells produced from a stem cell population discovered in the human RPE, the RPESC (see U.S. Pat. No. 8,481,313 for a detailed description of these cells and methods for their isolation and culture). Human RPESCs were used to generate human RPE having normal RPE morphology and physiology. The method used for culturing the RPE cells is described in detail in Blenkinsop et al. ((2013) Methods Mol Biol. 2013; 945:45-65). Briefly, primary adult RPE cells were obtained from cadaveric human eyes under IRB-approved protocols, within 36-hours of the time of death. The anterior half of the eye was removed followed by the vitreous, and retina, isolating the posterior eyecup with the RPE/Bruch's membrane/choroid complex intact (see Blenkinsop T A, et al.; Methods Mol Biol. 2013; 945:45-65; and Salero E, et al. Cell Stem Cell. 2012; 10(1):88-95). The RPE were removed and plated at a density of 100,000 cells/well in RPE medium (Maminishkis A, et al. Invest Ophthalmol Vis Sci. 2006; 47(8):3612-24) containing 10% fetal bovine serum (FBS). Once the primary (passage zero) cells reached confluence (˜20 days), the FBS concentration was reduced to 5%. Then, following the protocol developed by Salero S et al, 2013 (supra), the RPE were activated to proliferate in RPE medium with 5% FBS.

### Example 2

### TNFα and TNFβ Induce EMT in RPE Cells

This Example describes a screening assay for identifying agents that induce EMT.

The TNF locus possesses a strong genetic association with ERM in PVR. Hepatocyte growth factor has been found in PVR and is a known TNFα and TGFIβ activator. TGFβ levels are elevated in PVR vitreous samples and correlate with the growth of intraocular fibrotic ERM. Therefore both TNFα and TGFβ pathways have been individually implicated in ERM, but their combinatorial effects have not been tested. It is presently discovered that TGFβ and TNFα in combination have synergistic effects above and beyond their individual effects to induce proliferation, EMT, and ERM formation in epithelial cells. Thus, this combination of TGFβ and TNFα, termed “TnT,” was used to induce EMT in normal, healthy RPE cultures.

Following production of RPE cells with typical cobblestone morphology, as described in Example 1, a screening assay was performed to identify agents that disrupt the normal RPE morphology and physiology, and induce EMT.

RPE were plated at a confluency of 30,000 cells in 1 well of a 24-well plate in DMEM/F12 with 5% FBS, adding TGFβ (10 ng/ml) or TNFα (10 ng/ml), or both. After 5 days, RPE morphology was assessed by a light microscope.

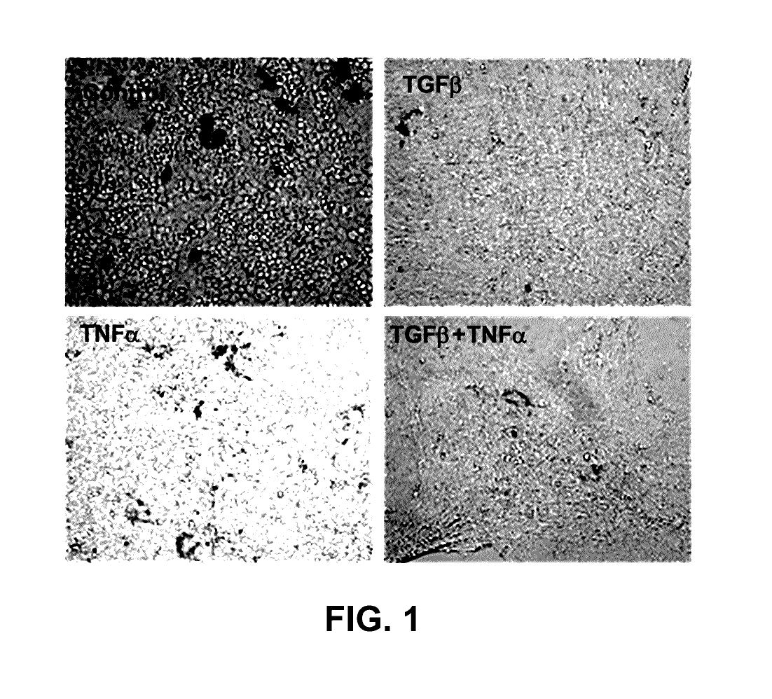

In control conditions, RPE cells formed a typical cobblestone epithelial monolayer, while in groups exposed to TGFβ or TNFα, the RPE cells lost epithelial morphology (FIG. 1). Further, in the presence of 10 ng/ml of TGFβ or TNFα, the RPE cells acquired a fibroblastic morphology, indicating a mild form of EMT (FIG. 1). TGFβ and TNFα in combination (“TnT”) resulted in more pronounced EMT, and the growth of three dimensional masses of cells, particularly surrounding the sides of the wells. Those masses resembled those found in advanced PVR, and were found in vitro exclusively in the TnT condition (FIG. 1).

### Example 3

### Identification of Cell Markers Associated with EMT

This Example describes the identification of EMT-associated gene transcripts in RPE cells, including SNAIL, SLUG, and TWIST, as well as the discovery that p38 undergoes nuclear translocation in RPE cells cultured in conditions that lead to EMT.

Gene transcripts associated with EMT were identified using quantitative real-time PCR. RNA was extracted at various time points, and assayed for EMT-related gene transcripts. Transcript quantification was normalized relative to RPE cultured for the same time points in vehicle controlled media.

EMT-related transcripts increased above control in the presence of the combination of TGFβ and TNFα. The EMT-associated transcripts SNAIL, SLUG and TWIST increased significantly over the course of 1-5 days particularly in the TnT condition.

TABLE 3
List of primers used for Real
Time PCR on adult human RPE

| | | | Product | |
|---|---|---|---|---|
| | | | Size | Tₐₙₙ |
| Human Gene | Forward 5′-3′ | Reverse 3′-5′ | (bp) | (° C.) |
| MITF (GenBank® | TTGTCCATCTGCCTC | CCTATGTATGACCAG | 87 | 55 |
| No NM_198178 | TGAGTAG | GTTGCTTG | | |
| | (SEQ ID NO: 40) | (SEQ ID NO: 41) | | |
| RPE65 (GenBank® | TGGTGTAGTTCTGAG | AGTCCATGAAAGGTG | 137 | 60 |
| No. NM_000329) | TGTGGTGGT | ACAGGGATGTT | | |
| | (SEQ ID NO: 42) | (SEQ ID NO: 43) | | |
| SNAIL (GenBank® | TGTCAGATGAGGACA | CTGAAGTAGAGGAGA | 611 | 53 |
| No. NM_005985) | GTGGGAAAGG | AGGACGAAGG | | |
| | (SEQ ID NO: 44) | (SEQ ID NO: 45) | | |
| SLUG (GenBank® | AGCGAACTGGACACA | TCTAGACTGGGCATC | 410 | 55 |
| No. NM_003068 | CATAC | GCAG | | |
| | (SEQ ID NO: 46) | (SEQ ID NO: 47) | | |
| TWIST (GenBank® | GTCCGCAGTCTTAGC | GCTTGAGGGTCTGAA | 156 | 60 |
| No. NM_000474) | AGGAG | TCTTGCT | | |
| | (SEQ ID NO: 48) | (SEQ ID NO: 49) | | |

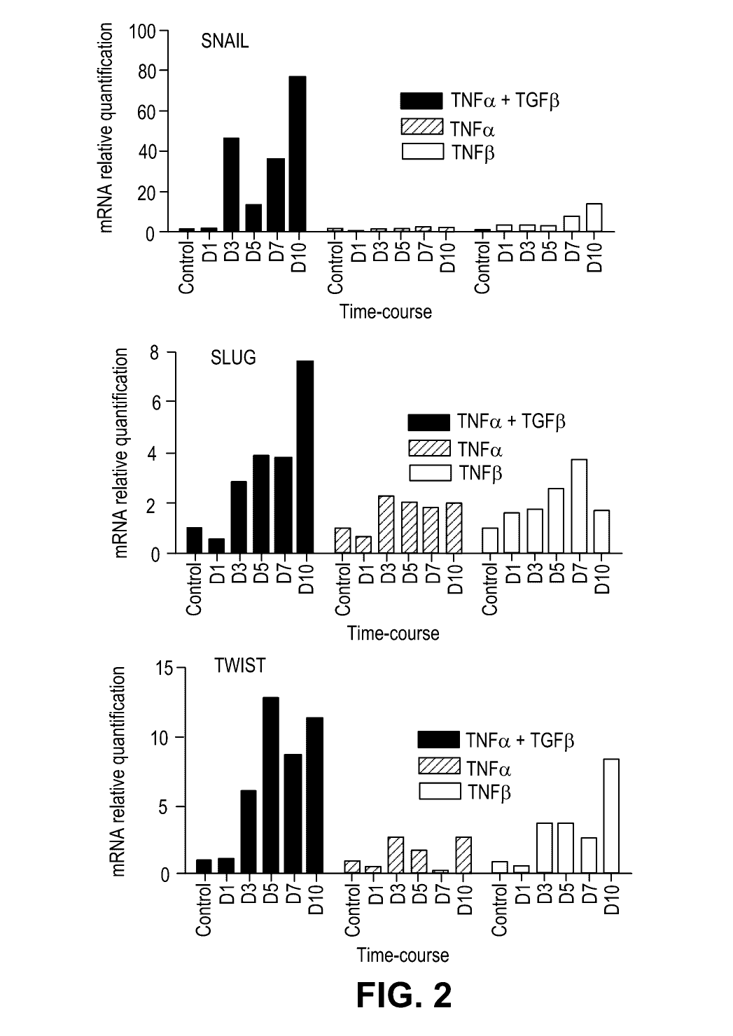

For SNAIL expression, the combination of TGFβ or TNFα (TnT) was synergistic, since the increase was markedly greater than the sum of the individual increases due to TNFβ or TNFα alone (FIG. 2).

The explosive effect of the TnT condition on SNAIL gene transcription suggests TGFβ and TNFα signaling pathways converge downstream to facilitate SNAIL transcription. A battery of signaling pathways were assayed to determine which was involved in the downstream gene expression effects in SNAIL, SLUG and TWIST. P38 stood out as having a particularly strong change in nuclear localization upon TnT application. Since the p38 signaling pathway is downstream of TGFβ and TNFα signaling, p38 activity in the RPE cells was studied. RPE cells were cultured in DMEM with 5% FBS in the presence of TGFβ and/or TNFα for 5 days then fixed and immunostained for p38 (Tocris).

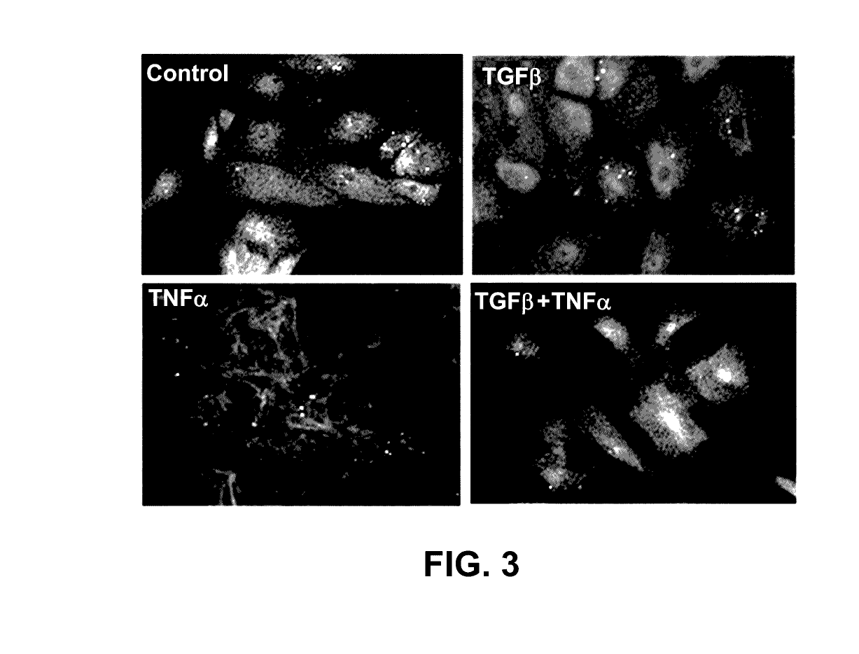

Under control and TGFβ conditions, p38 was localized predominantly perinuclearly, whereas p38 was found cytoskeletally in the TNFα condition. Interestingly, p38 localized nuclearly in the TnT condition, suggesting p38 has been activated to affect gene transcription (FIG. 3).

### Example 4

### p38 Blockade Blocks TnT-Induced EMT Gene Transcription and EMT

This Example demonstrates that p38 is upstream of EMT gene transcription and is in involved in EMT formation.

It was next reasoned that, if p38 is involved in the TnT-induced gene changes observed in SNAIL, SLUG and TWIST transcripts, then blocking p38 should result in the loss of TnT-induced EMT changes. Therefore an experiment was conducted to examine RPE-EMT with an additional condition of TnT+the p38 inhibitor SB202190.

RPE cultured for 5 days in DMEM with 5% FBS in the presence of 10 ng/ml TGFβ, 10 ng/ml TNFα and/or 10 ng/ml SB202190.

The TnT condition produced three dimensional masses and mRNA expression of SNAIL, SLUG, and TWIST increased significantly compared to all other conditions, as expected. In the condition where the RPE cells were cultured in the presence of TnT and SB202190, the three dimensional masses did not grow, and SNAIL, SLUG and TWIST transcription stayed at or near control levels (FIG. 4). Thus, the p38 inhibitor blocked RPE EMT and ERM formation.

### Example 5

### Identification of Novel Transcription Factor Involved in EMT

This Example demonstrates that FOXS1 is the upstream activator of SNAIL and SLUG in the TnT-induced EMT process.

RNA-seq of the EMT model was conducted under the following conditions: cobblestone control RPE, RPE exposed to TGFβ, TNFα, or TnT for 5 days. RNA was purified with Qiagen RNAeasy mini kit and tested for quality using the ND-1000 Nanodrop. RNA was converted to cDNA then amplified to double-stranded cDNA by NuGEN single primer isothermal amplification. cDNA was then fragmented into 300 base pair length using Covaris-S2 system and then end-repaired to generate blunt ends with 5′ phosphatase and 3′ hydroxyls and adapters were ligated for paired end sequencing on Illumina HiSeq 2000. RNA-Seq reads were aligned to the human genome (GRCh37/hg19) using the software TopHat.

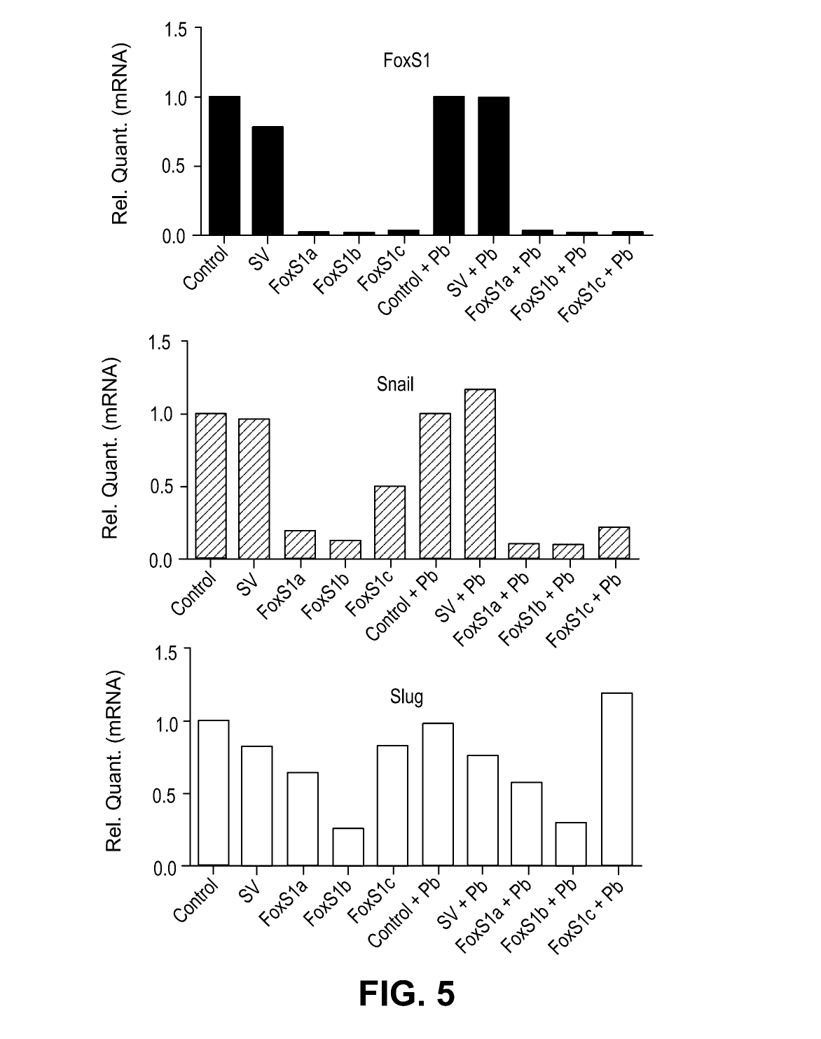

Gene transcripts found exclusively in the TnT condition were identified to find those involved in RPE EMT. The focus was on identifying transcription factors, since they regulate global gene transcription changes. FOXS1 was identified as a transcription factor that was uniquely expressed in the TnT condition (FIG. 5).

To study the role of FOXS1 in EMT, FOXS1 expression was inhibited using three knockdown constructs in RPE cells in which EMT was induced using TnT, and the effect on expression of the transcription factors SNAIL, SLUG and TWIST was determined. The following hairpin oligonucleotides were used:

FoxS1 shRNAa
(SEQ ID NO: 50)
(GCCAGGAATGTTCTTCTTTG);
FoxS1 shRNAb
(SEQ ID NO: 51)
(GCCAATAAAGCCATGTGAT);
and
FoxS1 shRNAc
(SEQ ID NO: 52)
(GCATCTACCGCTACATCAT).

The shRNAs were inserted into the FUGW-H1 lentiviral construct as described in the publication by Phoenix and Temple (Genes Dev. 2010 Jan. 1; 24(1):45-56). A scrambled set of oligonucleotides was inserted at the same location in the FUGW-H1 plasmid as negative control.

RPE was cultured with 10 ng/ml TGFβ and 10 ng/ml TNFα (TnT condition) for 5 days, in the presence of one of the knockdown constructs, control, or scrambled vector as control.

After 5 days gene transcription was quantified, and it was found that FOXS1 was efficiently knocked down by all three constructs. SNAIL had decreased expression when FOXS1 was knocked down, but not when the scrambled virus was introduced (FIG. 5). SLUG also had decreased expression to a smaller extent (FIG. 5). TWIST expression was determined as well, but no effect of FOXS1 knockdown was observed.

### Example 6

### p38 Activates FoxS1

This example demonstrates that p38 is upstream of FOXS1 in the RPE EMT model.

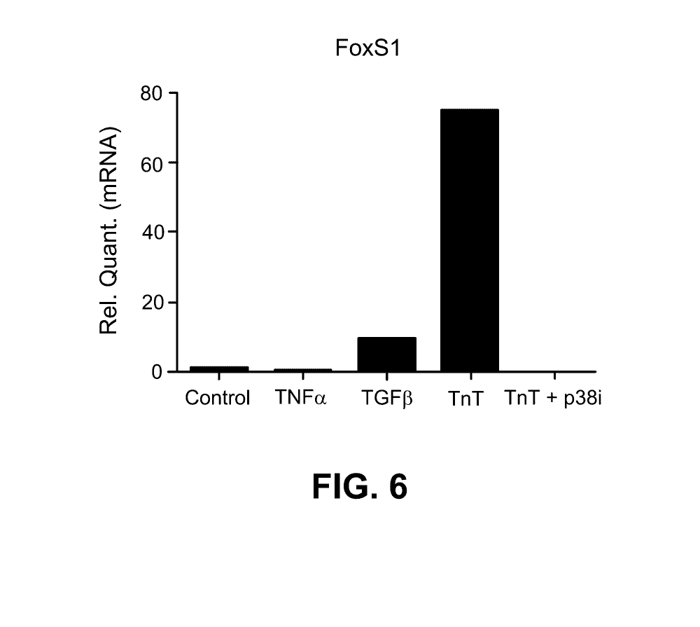

RPE cells were cultured in vitro in the presence of TnT and the p38 inhibitor SB202190 (“EMT+p38 inhibitor”), as described in Example 4. FOXS1 expression was measured in the EMT+p38 inhibitor condition after 5 days. FOXS1 gene transcription was elevated in the TnT condition, but not when the p38 inhibitor was added (FIG. 6). These data, in combination with the data in Example 5, indicate that FOXS1 is downstream of p38, and upstream of SNAIL and SLUG, show that the p38 pathway is active in EMT in RPE cells, and suggests that p38 activates FoxS1.

### Example 7

### FOXS1 is Sufficient to Induce EMT in RPE

This example demonstrates that FOXS1 overexpression induces increased expression of EMT-associated transcription factors SNAIL, SLUG and TWIST.

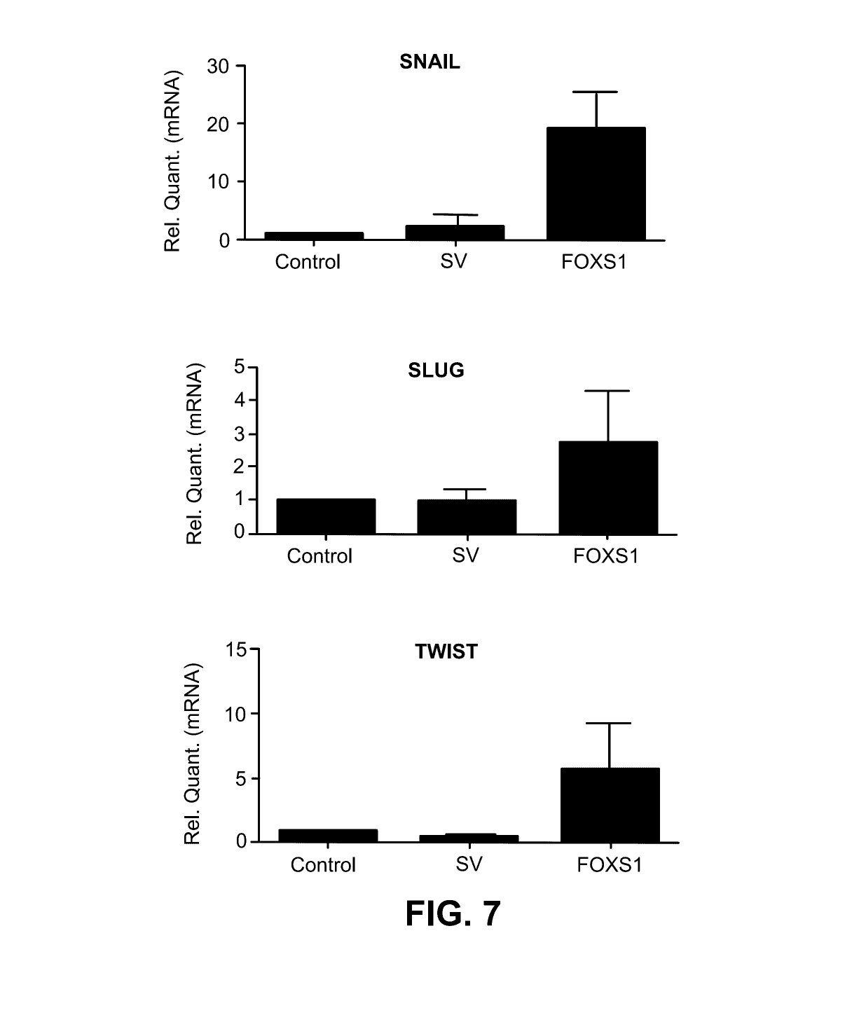

In order to determine whether FOXS1 can induce SNAIL and SLUG transcripts independent of p38 or TnT conditions, an overexpression lentiviral construct of FOXS1 was developed. RPE cells were cultured for 5 days in DMEM with 5% FBS and infected with a FOXS1 lentiviral overexpression construct. After 5 days gene transcription was assayed. As shown in FIG. 7, FOXS1 overexpression induced an increase in gene transcription in SNAIL, SLUG and TWIST. In sum, these results show that FOXS1 is necessary and sufficient to induce EMT in RPE.

### Example 8

### FOXS1 Drives EMT in Breast Epithelia

This example demonstrates that FOXS1 mediates the TGFβ signaling pathway and drives EMT in other, non-RPE epithelia such as breast, where EMT is known to result in abnormal tissue growth.

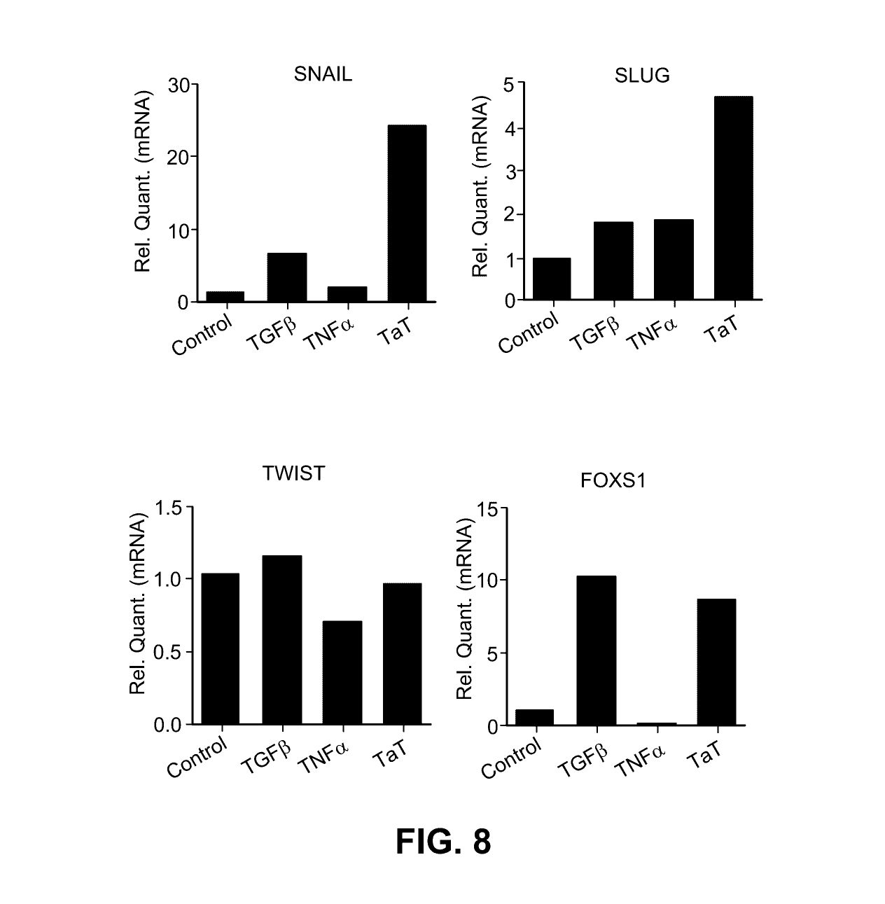

The involvement of FOXS1 in driving EMT in the human breast epithelial cell line hTERT HMEnt (HME) was investigated. HME cells were cultured in the presence of TGFβ, TNFα or both (TnT), as described in Example 2, and assayed for EMT and FOXS1 transcripts. FIG. 8 shows that, similar to data in RPE cells, both SNAIL and SLUG gene transcription increase predominantly in the TnT condition. Moreover, FOXS1 increased in both the TGFβ alone condition, and in the TnT condition, indicating that, although the role of FOXS1 is not identical with its role in RPE cells, FOXS1 mediates the TGFβ pathway and induces EMT in other human epithelia.

### Example 9

### FOXS1 is Associated with ERM in Human RPE

This example demonstrates that the pathways underlying EMT in the in vitro RPE model of ERM were also active in ERM surgically removed from patients.

The role for FOXS1 in ERM discovered in the Examples above was next confirmed in ERM taken directly from human patients.

RNA was isolated from surgically removed epiretinal membranes (ERM) taken from two patients with epiretinal membranes and macular pucker. The ERM transcripts in those ERM patients were compared to those in the RPE model.

In the human ERM samples, SNAIL transcript levels were determined to be in between levels for RPE and EMT-RPE, SLUG levels were close to cobblestone control levels. FOXS1 transcript quantities are in between RPE and EMT-RPE, as were MITF transcripts. OTX2 transcript level was below both RPE and EMT-RPE, while Bestrophin (“BEST”) transcript levels were higher than in either RPE or EMT-RPE.

The expression of MITF, OTX2, and Bestrophin in these ERMs implicated RPE as contributors to the ERM. These data further confirm that the same pathways are active in RPE EMT in vitro as found in patient ERMs, validating the RPESC-based in vitro model of ERM formation. Further, FOXS1 has been identified as a novel target for ERM therapy.

### Example 10

### Treatment of EMT in Human Patients

RPE undergo EMT in epiretinal membrane formation and inhibition of FOXS1 or p38 can be used in clinical studies to inhibit the process of RPE EMT and thereby treat ERM formation causing macular pucker, proliferative vitreoretinopathy, preretinal fibrosis, vitreomacular traction, tractional retinal detachment, and phthsis bulbi.

For example, groups of patients diagnosed with an EMT-associated disease are treated with a FOXS1 inhibitor as follows. FOXS1 antisense oligonucleotides confirmed in vitro to inhibit FOXS1 expression are administered to the retina of each patient by intavitreal injection. Some groups of patients receive a single administration, other groups receive multiple administrations over several weeks. The patient is monitored to determine if EMT is treated (e.g., by stabilization of the RPE phenotype and/or by reversing EMT and/or by inhibiting further EMT.

In other studies, treatment of individuals with ERM with an intraocular formulation of a FOXS1 inhibitor is compared to untreated individuals with ERM for U.S. Food and Drug Administration (FDA) evaluation.

Several dosages of the FOXS1 inhibitor are tested.

Patients are monitored for regression of symptoms of the EMT-associated disease.

Similar studies are carried out for other inhibitors of the FOXS 1 signaling pathway, and related studies are carried out in breast cancer patients and patients with other epithelial cancers.

### Example 11

### Nicotinaminde (NAM) Inhibits EMT Markers in RPE Cells

Cadaver donor globes were received from the National Disease Research Interchange (Philadelphia, Pa., USA), and RPE cells were isolated and grown to establish a RPE monolayer (Blenkinsop et al., (2015), Investigative Ophthalmology & Visual Science 56, 7085-7099; Blenkinsop et al., (2013), Methods Mol Biol 945, 45-65.).

Primary human RPE lines were cultured in the presence of Nicotinamide (NAM) (Cat. #N0636; Sigma) at a concentration of 10 mM or vehicle (cell culture grade water) for 21 days. Cells from NAM and vehicle treated wells were collected in RNA protect at day 21 and RNA was isolated. cDNA was synthesized from RNA. Expression of Epithelial to Mesenchymal (EMT) associated protein transcripts including FOXS1 in NAM and vehicle treated RPE was tested by qPCR. Additionally, RPE fate associated protein transcripts were tested by qPCR.

In a parallel experiment, the effect of NAM was tested in an EMT model. EMT was induced in RPE cells using 10 ng/ml TGFβ1 and TNFα. RPE cells were trypsinized and passaged into a 24 well plate. 24 hours after passaging, RPE cells were treated with TGFβ1 and TNFα (TNT condition) for 7 days. To test the effect of NAM, we added 10 mM NAM or vehicle to the medium used for EMT model (using TNT). EMT associated protein transcripts including FOXS1 were analyzed by qPCR.

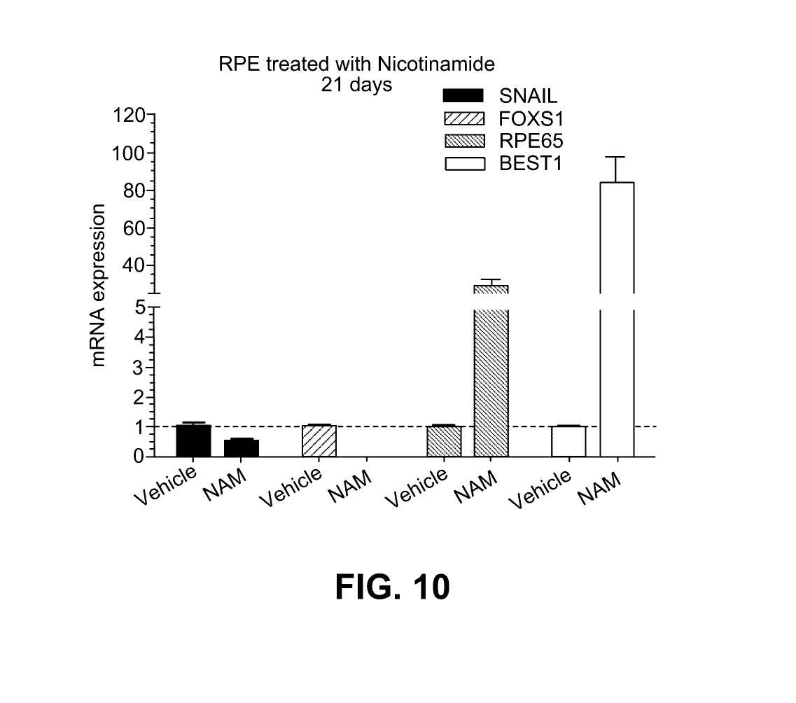

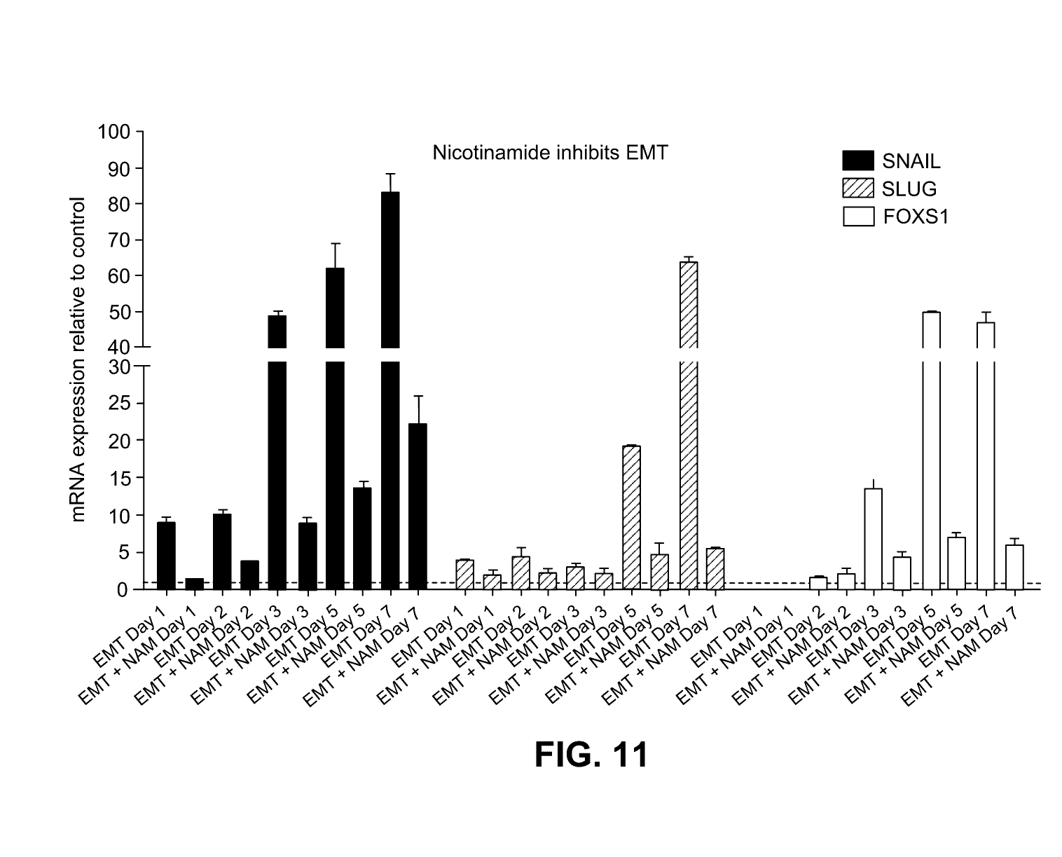

RPE cells cultured with NAM for 21 days showed increased expression of RPE markers-RPE65 and BEST1, while EMT associated markers SNAIL and FOXS1 were inhibited. (FIG. 10) Similarly, in the EMT model NAM inhibited EMT marker SNAIL as early as day 1 post induction of EMT. (FIG. 11) EMT markers FOXS1 and SLUG showed clear inhibition by day 3 and 5 respectively, post induction of EMT. (FIG. 11)

The results show that NAM preserves the RPE morphology by promoting the expression of RPE markers and inhibiting EMT markers such as FOXS1, even in the EMT model. Overall, it suggests that NAM can inhibit EMT in RPE cells.

All publications, patent applications, patents, GenBank® Accession Nos. and other references mentioned herein are incorporated by reference in their entirety. The materials, methods, and examples disclosed herein are illustrative only and not intended to be limiting.

A number of embodiments of the invention have been described. Nevertheless, it will be understood that various modifications may be made without departing from the spirit and scope of the invention. Accordingly, other embodiments are within the scope of the following claims.

What is claimed is:

1. A method for inhibiting epithelial to mesenchymal transition (EMT) in epithelial cells, which comprises contacting the epithelial cells with an inhibitor of the forkhead box s1 (FOXS1) signaling pathway.

2. The method of claim 1, wherein the inhibitor is a molecule selected from the group consisting of an antisense oligonucleotide, a small molecule, a peptide, and a ribozyme.

3. The method of claim 1, wherein the inhibitor is an antisense oligonucleotide selected from the group consisting of a double-stranded RNA (dsRNA) molecule or analogue thereof, a double-stranded DNA (dsDNA) molecule or analogue thereof, a short hairpin RNA molecule, and a small interfering RNA (siRNA) molecule.

4. The method of claim 1, wherein the inhibitor is an inhibitor of human FOXS1.

5. The method of claim 1, wherein the inhibitor targets a member of the p38 signaling pathway.

6. The method of claim 5, wherein the inhibitor is the small molecule p38 inhibitor SB202190.

7. The method of claim 1, wherein the epithelial cells are retinal pigment epithelial (RPE) cells.

8. The method of claim 1, wherein the epithelial cells are breast epithelial cells.

9. The method of claim 1, wherein the epithelial cells are RPE cells in a subject.

10. The method of claim 1, wherein the epithelial cells are cultured RPE cells.

11. The method of claim 1, wherein the epithelial cells are breast epithelial cells in a subject.

12. The method of claim 1, wherein the epithelial cells are cultured breast epithelial cells.

13. The method of claim 1, wherein the inhibitor is nicotinamide.

14. A method for treating a disease or disorder associated with EMT, wherein the method comprises administering to a subject in need of such treatment a composition comprising an inhibitor of the FOXS1 signaling pathway, wherein the inhibitor is present in an effective amount for decreasing FOXS1 expression in the subject.

15. The method of claim 14, wherein the disease or disorder is selected from the group consisting of epiretinal membrane formation (ERM), proliferative vitreoretinopathy (PVR), and macular pucker.

16. The method of claim 14, wherein the disease or disorder is abnormal breast epithelial cell growth.

17. The method of claim 16, wherein the abnormal breast epithelial cell growth is breast cancer.

18. The method of claim 14, wherein the inhibitor is present in an amount effective for decreasing the expression of one or more of SNAIL, SLUG, and TWIST in the subject.

19. The method of claim 14, further comprising measuring the expression level of FOXS1 in the subject.

20. The method of claim 19, wherein the expression level of FOXS1 is measured in a surgically removed tissue affected by EMT.

21. The method of claim 18, further comprising measuring the expression level in the subject of one or more of SNAIL, SLUG, and TWIST.

22. The method of claim 21, wherein the expression level of the one or more of SNAIL, SLUG, and TWIST is measured in a surgically removed tissue sample affected by EMT.

23. The method of claim 15, wherein the inhibitor is present in an amount effective for increasing the expression of one or both of OTX2 and Bestrophin in the subject.

24. The method of claim 23, further comprising measuring the expression level in the subject of one or both of OTX2 and Bestrophin.

25. The method of claim 24, wherein the expression level of one or both of OTX2 and Bestrophin is measured in a surgically removed retinal tissue sample affected by EMT.

26. The method of claim 14, wherein the inhibitor is a molecule selected from the group consisting of an antisense oligonucleotide, a small molecule, a peptide, and a ribozyme.

27. The method of claim 26, wherein the inhibitor is an antisense oligonucleotide selected from the group consisting of a double-stranded RNA (dsRNA) molecule or analogue thereof, a double-stranded DNA (dsDNA) molecule or analogue thereof, a short hairpin RNA molecule, and a small interfering RNA (siRNA) molecule.

28. The method of claim 27, wherein the inhibitor is an inhibitor of human FOXS1.

29. The method of claim 14, wherein the inhibitor targets a member of the p38 signaling pathway.

30. The method of claim 29, wherein the inhibitor is the small molecule p38 inhibitor SB202190.

31. The method of claim 14, wherein the epithelial cells are RPE cells.

32. The method of claim 14, wherein the inhibitor is nicotinamide.

33. A method of screening for a compound that inhibits EMT in epithelial cells, wherein the method comprises:
(a) providing a monolayer of epithelial cells;
(b) culturing the monolayer of cells in conditions that induce the cells to undergo EMT;
(c) contacting the monolayer of cells with a test compound;
(d) determining the expression level of at least one member of the FOXS1 signaling pathway; and
(e) identifying the test compound as a candidate inhibitor of EMT if the expression level of the at least one member of the FOXS1 signaling pathway is decreased relative to a control or a reference level.

34. A method of screening for a compound that inhibits EMT in epithelial cells, wherein the method comprises:
(a) providing a monolayer of epithelial cells;
(b) culturing the monolayer of cells in conditions that induce the cells to undergo EMT;
(c) contacting the monolayer of cells with a test compound;
(d) determining the expression level of one or more of the EMT-associated markers selected from the group consisting of FOXS1, SLUG, SNAIL, and TWIST; and
(e) identifying the test compound as a candidate inhibitor of EMT if the expression level of the one or more markers is decreased relative to a control or reference level.

35. The method of claim 33, wherein the epithelial cells are RPE cells.

36. The method of claim 33, wherein the epithelial cells are breast epithelial cells.

37. The method of claim 33, further comprising measuring the expression level of one or both of OTX2 and Bestrophin.

38. The method of claim 33, wherein the at least one member of the FOXS1 signaling pathway is selected from the group consisting of FOXS1 and p38.

39. The method of claim 33, wherein culturing the cells under conditions that induce EMT comprises contacting the cells with one or both of TNFα and TGFβ.

40. The method of claim 33 wherein the expression level is gene expression level.

41. The method of claim 40, wherein the gene expression level is measured using quantitative real-time polymerase chain reaction (PCR).

42. The method of claim 33, wherein the expression level is protein expression level.

43. The method of claim 42, wherein the protein expression level is determined using an assay selected from the group consisting of immunoblot, immunohistochemistry, fluorescence microscopy, ELISA, and multiplex assay.

44. A pharmaceutical formulation comprising: (a) an effective amount for inhibiting the FOXS1 signaling pathway of an inhibitor selected from the group consisting of an antisense oligonucleotide, a small molecule, a peptide, and a ribozyme, and (b) a pharmaceutical carrier; wherein the inhibitor reduces FOXS1 expression level and/or activity when administered to a subject suffering from EMT.

45. The formulation of claim 44, wherein the formulation is for use in the treatment of a disease or disorder associated with EMT.

46. The formulation of claim 44, wherein the disease or disorder is selected from the group consisting of ERM formation, macular pucker, and PVR.

47. The formulation of claim 44, wherein the disease or disorder is abnormal breast epithelial cell growth.

48. The formulation of claim 47, wherein the abnormal breast epithelial cell growth is breast cancer.

49. The formulation of claim 44, wherein the inhibitor is an inhibitor of FOXS1.

50. The formulation of claim 44, wherein the inhibitor is an inhibitor of the p38 signaling pathway.

51. The formulation of claim 50, wherein the inhibitor inhibits p38.

52. The formulation of claim 51, wherein the inhibitor is the small molecule p38 inhibitor SB202190.

53. The formulation of claim 44, wherein the inhibitor is nicotinamide.
